(19) United States
(12) Patent Application Publication
(10) Pub. No.: US 2023/0355708 A1
(43) Pub. Date: Nov. 9, 2023

(54) COMPOSITIONS FOR INHIBITING VIRAL ENTRY AND METHODS USING SAME

(71) Applicant: Drexel University, Philadelphia, PA (US)

(72) Inventors: Irwin M. Chaiken, Gladwyne, PA (US); Aakansha Nangarlia, Newtown, PA (US); Gabriela Canziani, Philadelphia, PA (US); Cameron Frank Abrams, Philadelphia, PA (US); Adel Ahmed Rashad Ahmed, Philadelphia, PA (US); Mark R. Contarino, Philadelphia, PA (US); Bibek Parajuli, Philadelphia, PA (US)

(21) Appl. No.: 18/029,032

(22) PCT Filed: Sep. 29, 2021
(86) PCT No.: PCT/US2021/052609

§ 371 Date: Mar. 28, 2023

Related U.S. Application Data
(60) Provisional application No. 63/189,424, filed on May 17, 2021.
(60) Provisional application No. 63/087,459, filed on Oct. 5, 2020.
(60) Provisional application No. 63/084,961, filed on Sep. 29, 2020.

(51) Int. Cl. A61P 31/14; A61K 47/60; A61K 38/08; C07K 7/60; A61P 31/18; A61K 38/16; A61K 47/65; A61K 45/06

(52) U.S. Cl. CPC A61K 38/164; A61K 38/08; A61K 38/16; A61K 38/162; A61K 45/06; A61K 47/60; A61K 47/65; A61P 31/14; A61P 31/18; C07K 7/60; A61K 38/00

(57) ABSTRACT

The present disclosure relates, in part, to a composition for promoting virolysis and/or inhibition of infection of a vims in a mammal, the composition comprising a lectin mutant. In certain embodiments, the lectin mutant comprises mutant cyanovirin N (CVN) and mutant Griffithsin (GRFT). The present disclosure further provides methods of treating, preventing, and/or ameliorating viral infection in a subject. In certain embodiments, the viral infection is caused by a vims selected from the group consisting of SARS-CoV-1, SARS-CoV-2, and HIV-I. The present disclosure further provides cyclic compounds useful for the treatment, prevention, and/or amelioration of HIV-I in a subject.--

### CROSS REFERENCE TO RELATED APPLICATIONS

This application claims priority under 35 U.S.C. § 119(e) to U.S. Provisional Application No. 63/084,961, filed Sep. 29, 2020, U.S. Provisional Application No. 63/087,459, filed Oct. 5, 2020, and U.S. Provisional Application No. 63/189,424, filed May 17, 2021, each of which are incorporated herein by reference in their entireties.

### SEQUENCE LISTING

The ASCII text file named “046528-7111WO1” created on Sep. 27, 2021, comprising 12.0 Kbytes, is hereby incorporated by reference in its entirety.

### BACKGROUND OF THE INVENTION

Viruses pose an ongoing threat to society. The human immunodeficiency virus-1 (HIV-1) is responsible for a global Acquired ImmunoDeficiency Syndrome (AIDS) epidemic, with over 33 million infected people worldwide. More recently, severe acute respiratory syndrome coronavirus 2 (SARS-CoV-2), the strain of coronavirus that causes coronavirus disease 2019 (COVID-19), has triggered an ongoing pandemic. The COVID-19 outbreak was declared a public health emergency on Jan. 30, 2020, and a pandemic on Mar. 11, 2020, by the World Health Organization (WHO).

Thus, there is a need in the art for compositions and methods for the treatment, prevention, and/or amelioration of viral infections, including but not limited to SARS-CoV-2 (COVID-19 disease) and/or HIV-1 (AIDS) in a human subject, and the present disclosure addresses this need.

### BRIEF SUMMARY OF THE INVENTION

The present disclosure provides a composition for promoting virolysis and/or inhibition of infection of a virus in a mammal, the composition comprising a lectin mutant selected from the group consisting of mutant cyanovirin N (CVN) and mutant Griffithsin (GRFT). In certain embodiments, the composition further comprises a S2 binding domain, wherein the lectin mutant and the binding domain are covalently linked by a flexible linker. In certain embodiments, binding domain comprises a HIV-1 MPER or MPER-like Trp3 domain.

The present disclosure further provides a pharmaceutical composition comprising at least one composition of the present disclosure and at least one pharmaceutically acceptable carrier.

The present disclosure further provides a method of treating, preventing, and/or ameliorating a viral infection in a subject, the method comprising administering to the subject a therapeutically effective amount of a compound of the present disclosure, which is optionally formulated as a composition and/or a pharmaceutical composition. In certain embodiments, the viral infection is caused by a coronavirus. In certain embodiments, the coronavirus is selected from the group consisting of SARS-CoV-1 and SARS-CoV-2. In certain embodiments, the viral infection is caused by HIV-1.

The present disclosure further provides a method of promoting virolysis of a virus in a subject, the method comprising administering to the subject in need thereof a therapeutically effective amount of a compound of the present disclosure, which is optionally formulated as a composition and/or a pharmaceutical composition. In certain embodiments, the virus is a coronavirus. In certain embodiments, the coronavirus is selected from the group consisting of SARS-CoV-1 and SARS-CoV-2. In certain embodiments, the virus is HIV-1.

The present disclosure further provides a cyclic compound of formula (I), or a salt, solvate, enantiomer or diastereoisomer thereof, wherein the substituents are defined elsewhere herein:

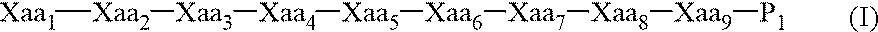

The present disclosure further provides a method of treating, preventing, and/or ameliorating HIV-1 infection in a subject in need thereof, the method comprising administering to the subject a therapeutically effective amount of at least one cyclic compound of the present disclosure or the pharmaceutical composition of the present disclosure.

### BRIEF DESCRIPTION OF THE DRAWINGS

The drawings illustrate generally, by way of example, but not by way of limitation, various embodiments of the present application.

FIG. 1A shows a comparison of spike proteins HIV-1 and SARS-CoV-2. FIG. 1B shows a comparison of conformation states of SARS-CoV-2 spike with different orientations of the ACE2 receptor binding domain of SARS-CoV-2 spike S1 subunit; (Left) Closed trimeric S ectodomain (PDB 6VXX); (Right) Trimeric S trimer ectodomain with one RBD up (6VYB) with overlay of RDB+ACE2 complex (PDB 6M0J). Spheres denote N-linked glycoslylation sites, with NAG’s resolved, located in both the S1 subunits and S2 subunits. The S2 subunit contains the preponderance of the high mannose glycan sites of the spike.

FIGS. 2A-2C show a comparison of lytic inactivation of HIV-1 and SARS-CoV-2 by lectin compositions. FIG. 2A shows lytic inactivation of HIV-1 by lectin-DLI denoted CVN-Lx-Trp3. FIG. 2B shows lytic inactivation of SARS-CoV-2 by CVN. FIG. 2C shows a schematic drawing of CVN-Lx-Trp3 DLIs originally reported, namely CVN-L8-His6-MPER and CVN-L3-Trp3.

FIGS. 3A-3E show validation of the surface plasmon resonance (SPR) SARS subunit platform.

FIGS. 4A-4D show domain specificity of lectin binding. FIG. 4A shows a schematic representation of the SPR binding assay with S1 and S2 domains. FIG. 4B and FIG. 4C show dose response plots as a function of time of CVN and MVN injections over S1 and S2 at concentrations spanning 4.4 to 350 nM, with 3-fold dilutions, respectively; a gp120 surface was used as a positive control. FIG. 4D shows a mapping of spike N-linked glycans on the prefusion structure of trimeric SARS-CoV-2 spike (PDB ID 6VSB). Oligomannose-type glycans comprise 32% of the total glycan pool and are dispersed across both S1 and S2 spike subunits.

FIG. 5 shows lectin binding specificity screening and kinetics of spike, including that the oriented spike and domains determine lectins glycan specificity, the soluble trimer configuration, and the S-spike trimer in the context of the lentivirus CoV Env.

FIGS. 6A-6B show the effect of CVN and CVN-L4-Trp3 on the SARS-CoV-2 pseudoviruses. FIG. 6A shows inhibition of SARS-CoV-2 infection with both the lectin, CVN, and lectin-DLI, CVN-L4-Trp3 as compared to Bt-Alkyne-L3-Trp4. FIG. 6B shows inactivation of viruses via cell free virolysis with both CVN and CVN-L4-Trp3 as compared to Bt-Alkyne-L3-Trp3 alone. Furthermore, CVN was more potent than CVN-DLI in virolysis.

FIGS. 7A-7C show the effect of CVN and CVN-L4-Trp3 to inhibit infection and cause virolysis across various virus strains. FIG. 7A shows inhibition of HIV-1 and SARS-CoV-1 infection with CVN similarly as with SARS-CoV-2. FIG. 7B shows no virolytic effect by CVN for HIV-1, Env Neg, and SARS-CoV-1 as compared to SARS-CoV-2. FIG. 7C shows a summary of the effects of CVN, CVN-L4-Trp3, and MVN across the four pseudovirus types. The symbols indicate positive effect, +, negative effect, -, and moderate or low-level effect, (+).

FIGS. 8A-8C show inhibition of infection and virolysis of SARS-CoV-2 by CVN, wherein CVN inhibits infection of SARS-CoV-2, SARS-CoV-1, and BaL01, and CVN causes virolysis of SARS-CoV-2; the potency of leakage is less for BaL01 and SARS-CoV-1.

FIGS. 9A-9B show inhibition of infection and virolysis of SARS-CoV-2 by CVN, wherein CVN inhibits infection of SARS-CoV-2, SARS-CoV-1, and BaL01, and CVN causes virolysis of SARS-CoV-2; the potency of leakage is less for BaL01 and SARS-CoV-1.

FIG. 10 shows the irreversible inactivation of SARS-CoV-1 and SARS-CoV-2 by CVN.

FIGS. 11A-11H show that CVN binds to both the S1 and S2 subunits of the SARS-CoV-2 spike protein, wherein the stoichiometry of binding is higher to the S2 subunit.

FIGS. 12A-12B show the effect of P5 1G-CVN on SARS-CoV-2 pseudoviruses. FIG. 12A: decreased inhibition of infection of SARS-CoV-2 by P51G as compared to CVN; FIG. 12B: reduced virolysis of SARS-CoV-2 by P51G-CVN.

FIGS. 13A-13H show that direct binding of P5 1G-CVN to SARS-CoV-2 spike subunits occurs, but with reduced affinity as compared to CVN.

FIGS. 14A-14C show that Griffithsin (GRFT) binds to SARS-CoV-2 spike subunits; GRFT binds to both S1 and S2 subunits of SARS-CoV-2 pseudoviruses, wherein the stoichiometry is higher for the S2 subunit as compared to the S1 subunit.

FIGS. 15A-15B show the effect of GRFT on SARS-CoV-1 and SARS-CoV-2 pseudoviruses. FIG. 15A: reduced inhibition of infection by SARS-CoV-1 and SARS-CoV-2 pseudoviruses; FIG. 15B: GRFT does not causes significant virolysis of SARS-CoV-1 and SARS-CoV-2 pseudoviruses.

FIGS. 16A-16C show greatly reduced binding of microvirin lectin (MVN) to SARS-CoV-2 spike subunits as compared to CVN and GRFT.

FIGS. 17A-17B show the effect of MVN on SARS-CoV-1 and SARS-CoV-2 pseudoviruses. FIG. 17A: MVN does not inhibit infection of SARS-CoV-1 or SARS-CoV-2 pseudoviruses; FIG. 17B: MVN does not cause virolysis of SARS-CoV-1 or SARS-CoV-2 pseudoviruses.

FIGS. 18A-18B shows irreversible inactivation of SARS-CoV-1 and SARS-CoV-1 with GRFT, but not with MVN.

FIG. 19 shows redesign of modular components of lectin type, linker length, and MPER-like Trp3 to derive enhanced-function lectin-DLIs.

FIG. 20 provides background on optimization efforts toward peptide macrocycles with greater potency, wherein macrocycle AAR029B (CD4 & 17b binding IC₅₀ = ~30 nM; gp120 shedding EC₅₀ = ~0.3 µM, inhibition of HIV-1 cell infection IC₅₀ = ~0.2 µM, and protease resistance (T_{½} > 3 h in rats) is optimized to AAR029N2 (HIV_{Bal.01} IC₅₀ = ~100 nM, HIV_{JRFL} IC₅₀ = ~112 nM; Fully-infected HIV_{NL4.3} IC₅₀ = ~180 nM; which binds clade_AE envelope without cytotoxicity).

FIG. 21 provides diagram showing the envisioned activity of cyclic peptide triazoles (cPT) HIV-1 killers, wherein dual-inhibition of envelope CD4 and co-receptor binding sites, irreversible Env self-destruction, and inactivation of Env on viruses and cells are achieved.

FIG. 22 show the efficacy of cyclic peptide triazole AAR029N2; HIV-1 viral load normalized to Hu CD4* T cell counts: data show significant efficacy in an animal model.

FIG. 23 shows the identification of 29N8 as a better lead compound than AAR029N2.

FIGS. 24A-24B shows improved inhibition of 29N8 with T-cells pre-infected by HIV-1 (fully infectious virus).

### DETAILED DESCRIPTION OF THE INVENTION

Almost four decades ago, the HIV-1 pandemic swept across human populations, infecting 60 million people, and causing over 25 million deaths globally. Today, HIV-1 is one of the leading causes of death in the world. The highly active antiretroviral therapy (HAART) has allowed us to prolong our patients’ lives and control the disease’s transmission, but it is no cure. To find a cure for HIV 1 and eradicate it, drugs need to target both infected cells and HIV-1 viruses.

Accordingly, HIV-1 envelope-targeting irreversible inactivators have been devised. These envelope inactivators target the HIV-1 envelope protein, gp120, and cause self-destruction of the HIV-1 envelope in both infected cells and viruses. Their unique modes of action make them unique in the market. Non-limiting examples of envelope inactivators include macrocyclic peptide triazoles, macrocyclic peptide triazole thiols, small CD4-mimetic dual action lytic inactivators, and lectin-dual action lytic inactivators. Each of these compounds has a unique mode of action but are all capable of causing irreversible inactivation.

For example, in one aspect, the present disclosure provides cyclic peptides suitable for inhibiting viral entry. The entry of HIV-1 into the host cell is mediated by interaction of a trimeric gp120/gp41 envelope (Env) protein complex with both cellular CD4 and chemokine co-receptor CCR5 or CXCR4. Each virus Env spike consists of a trimer of two non-covalently associated glycoproteins, an inner gp41 transmembrane protein and an outer gp120 protein. The first step of viral entry is the interaction with CD4, leading to structural changes in the virus Env spike and exposing the chemokine binding domains of gp120. A structural change in the envelope spike exposes the fusion peptide sequence of gp41 and enables the collapse of gp41 into a six-helix bundle, leading to downstream membrane fusion and productive infection.

A class of triazole conjugated cyclic peptides are described herein. The compounds described herein have enhanced binding affinity for HIV-1 gp120, and block both CD4 and co-receptor sites with great efficacy. Further, the compounds appear to trap the gp120 protein in a non-functional state, distinct from the flexible ground state of gp120 or the CD4 induced conformation, and thus effectively halt the entry process at the initial binding stages.

Further, at the onset of the COVID-19 pandemic, the structural similarities between HIV-1 and SARS-CoV-2 viruses were observed. The SARS-CoV-2 virus uses the spike protein complex on the virus surface as a molecular machine to bind host cell receptor and mediate membrane fusion to enter the cells. Structurally inactivating such spikes was reasoned to prevent the fusion mechanism and stop infection. In one aspect, the structural metastability of the spike protein complex can be targeted for irreversible inactivation of the virus, and In certain embodiments, lectin-based proteins can be used to irreversible inactivate the spike protein complex of SARS-CoV-2.

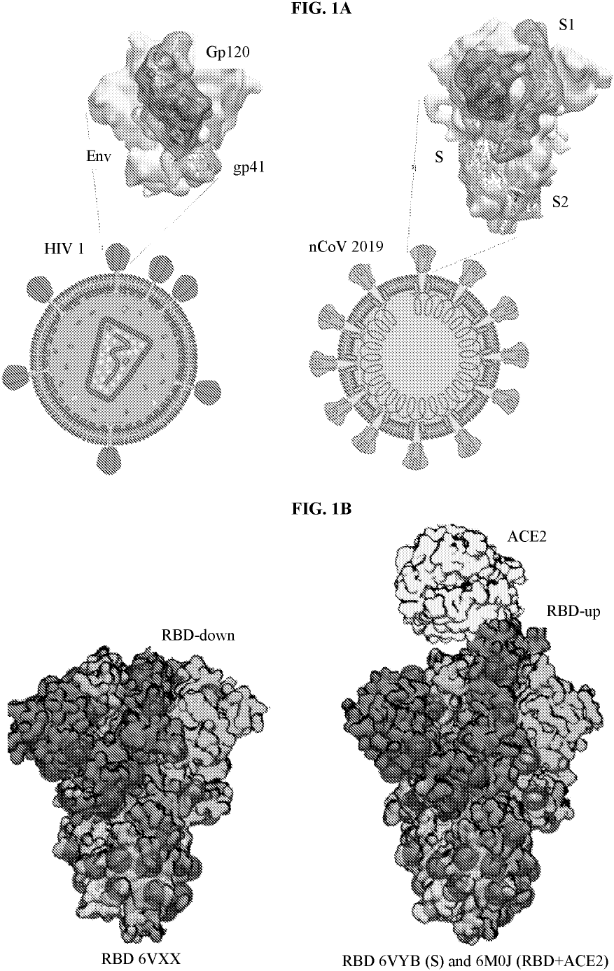

The hypothesis of targeting spike metastability for irreversible inactivation is underpinned by the realization that the SARS-CoV-2 spike is structurally homologous to HIV-1 Env spike (FIG. 1A). A large-scale conformational change program stabilized by ACE2 receptor interaction is built into the intrinsically unstable spike structure of SARS-CoV-2, with the receptor binding domain (RBD) swinging from a more hidden state to a more exposed receptor-interacting conformational state (FIG. 1B). The HIV-1 envelope possesses a metastability that allows for a cascade of receptor interactions and consequent conformational rearrangements enabling HIV-cell membrane fusion and entry. Importantly, agents have been identified that can utilize the conformational deformability of HIV-1 Env to stabilize different conformational states and can drive the Env into irreversibly inactivated end points. In certain embodiments, the intrinsic conformational metastability of SARS-CoV-2 provides a similar vulnerability to trigger irreversibly disordered states, hence irreversibly inactivating the spike complex, and consequently the virus, before cell encounter.

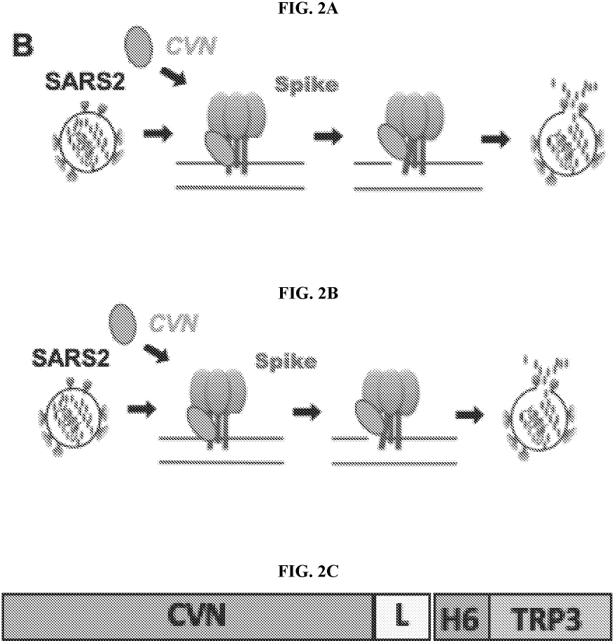

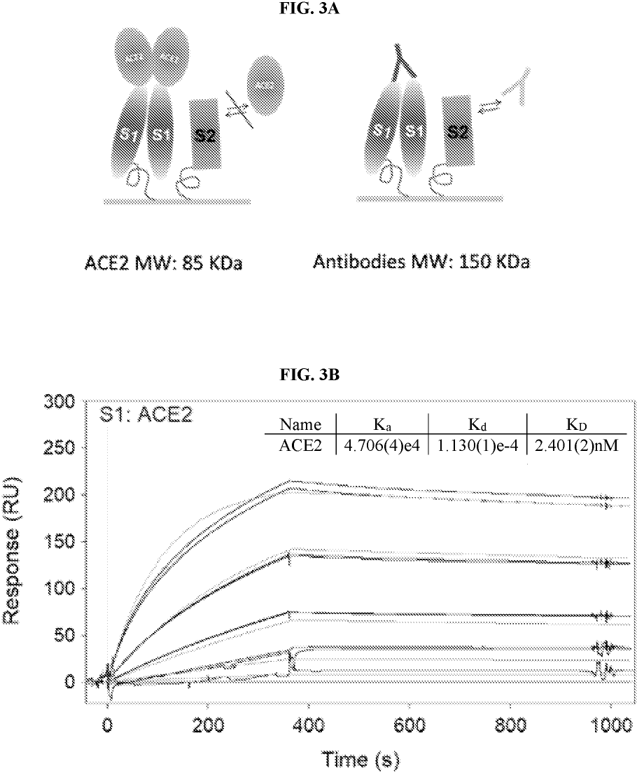

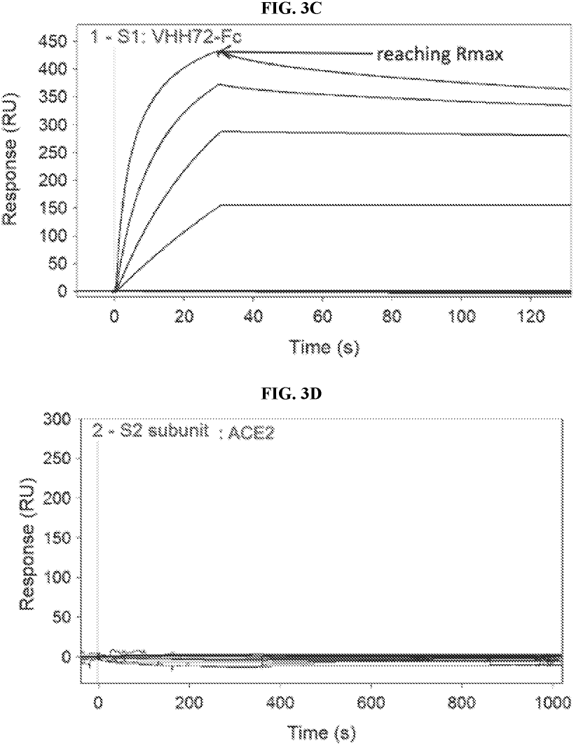

Dual-action Lytic Inhibitors (DLIs) are genetically engineered proteins whose hallmark is the capacity to hijack the metastable HIV-1 Env protein complex to cause irreversible disruption of the Env-containing membrane, leading to complete inactivation of viruses and Env-expressing cells. The first-generation DLI (formerly denoted ‘DAVEI’) was a recombinant fusion of the lectin cyanovirin-N (CVN) and HIV-1 MPER sequence, joined by a flexible linker. The CVN targets glycans on gp120 while the MPER peptide targets gp41, with dual engagement of both gp120 and gp4 1 required for lytic activity (FIGS. 2A-2B). Further, the DLI paradigm is both modular and optimizable, as demonstrated by using gp120 binders other than CVN and drastically minimized gp41-interacting components, typified by the fully active 9-residue “Trp3” peptide fragment of the membrane proximal external region (MPER) of gp41.

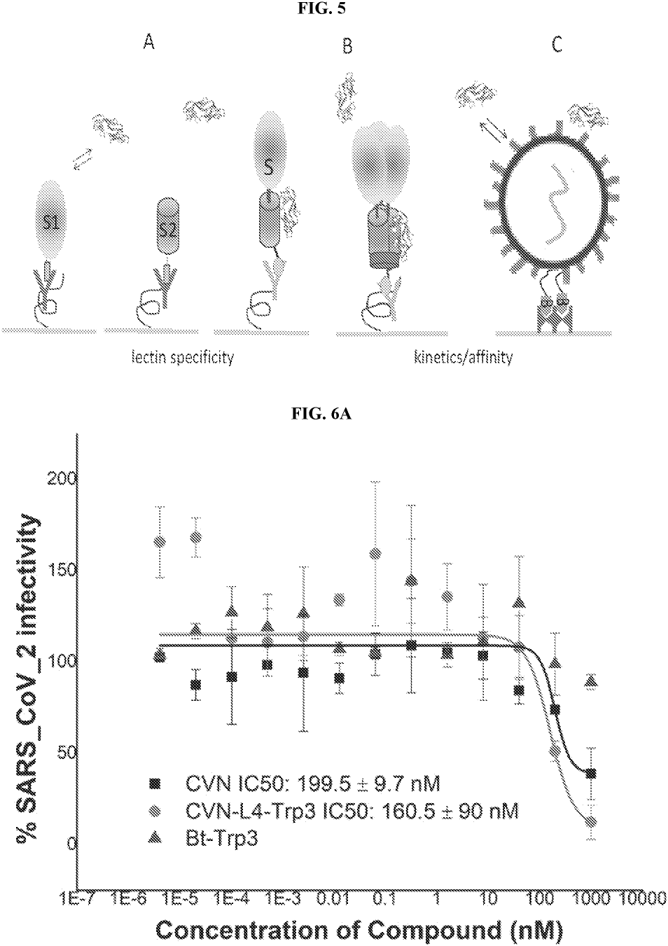

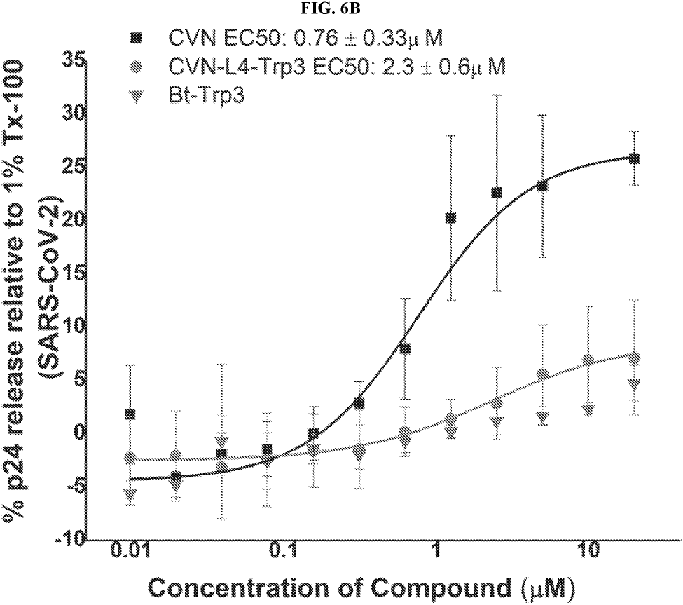

Coronavirus spike proteins, like the Env protein of HIV-1, are heavily glycosylated and have an MPER region with some homology to that of Env. In certain embodiments, the DLI platform can functionally inactivate the SARS-CoV-2 spike in much the same way that it does HIV-1 Env. Indeed, results show that the lectin cyanovirin-N(CVN)-DLI can potently inhibit SARS-CoV-2 pseudovirus infection of an ACE2-expressing cell line (FIGS. 6A-6B). It was found that the DLI neutralizing activities were associated with virolysis of SARS-CoV-2 pseudoviruses.

In one aspect, the present disclosure relates to the unexpected discovery that lectin CVN alone, caused irreversible inactivation of both SARS-CoV-1 and SARS-CoV-2 pseudoviruses in in vitro assays. Upon testing the effects of other lectins on SARS-CoV pseudoviruses, partial irreversible inactivation with GRFT was observed. These results suggest that the irreversible inactivation of virus is possible due to glycan engagement that disrupts the spike-membrane structure, perhaps due to spike conformational metastability. Furthermore, it has been reasoned that the irreversible inactivation function of CVN may be generally applicable to SARS-CoV. Overall, the results suggest that glycans are potential targets for development of treatments for SARS-CoV-2/COVID-19.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “about” as used herein can allow for a degree of variability in a value or range, for example, within 10%, within 5%, or within 1% of a stated value or of a stated limit of a range, and includes the exact stated value or range.

As used herein, the term “antiviral agent” means a composition of matter that, when delivered to a cell, is capable of preventing replication of a virus in the cell, preventing infection of the cell by a virus, or reversing a physiological effect of infection of the cell by a virus. Antiviral agents are well known and described in the literature. By way of example, AZT (zidovudine, Retrovir®, Glaxosmithkline, Middlesex, UK) is an antiviral agent that is thought to prevent replication of HIV in human cells.

As used herein, the term “βAla” or “bAla” refers to beta-alanine or 3-aminopropionic acid.

As used herein with respect to the compounds of the invention, “biologically active” means that the compounds elicit a biological response in a mammal that can be monitored and characterized in comparison with an untreated mammal. One possible biological response within the invention relates to the ability of the compound to treat, prevent, and/or ameliorate SARS-CoV-1, SARS-CoV-2, and/or HIV-1 infection in a mammal. In this particular case, the compound is administered to the mammal by an administration route selected from the group consisting of nasal, inhalational, topical, oral, buccal, rectal, pleural, peritoneal, vaginal, intramuscular, subcutaneous, transdermal, epidural, intratracheal, otic, intraocular, intrathecal and intravenous. The mammal and the viral load level in its body are monitored as a function of time, and the observation of a measurable and dose-dependent change in infection rate or viral load in the body is evidence that the compound displays biological activity. This preferred biological response does not limit or restrict the disclosures or embodiments of the invention in any way.

The term “binding domain” as used herein refers to a amino acid and/or polypeptide sequence, or fragment thereof, which binds to a molecule of interest, including but not limited to small molecules, proteins (e.g., enzymes and/or receptors), DNA, and/or RNA. In certain embodiments, the binding domain is a S2 binding domain, wherein the binding domain binds to the S2 subunit of the SARS-CoV-1 and/or SARS-CoV-2 spike protein.

As used herein, the term “CM5” refers to carboxymethyl dextran.

As used herein, the term “container” includes any receptacle for holding the pharmaceutical composition. For example, In certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound’s ability to perform its intended function, e.g., treating, ameliorating, or preventing shivering in a subject.

As used herein, the term “Dab” or “Dbu” refers to 2-diaminobutyric acid.

As used herein, the term “DCM” refers to dichloromethane.

As used herein, the term “Dde” refers to the protective group 1-(4,4-Dimethyl-2,6-dioxocyclohex-1-ylidene)ethyl.

As used herein, the term “DIC” refers to N,N′-diisopropylcarbodiimide.

As used herein, the term “DIPEA” refers to N,N-diisopropyl-ethylamine.

As used herein, the term “Dmab” refers to the protective group 4-(N-[1(4,4-Dimethyl-2,6-dioxocyclohexylidene)-3-methylbutyl] amino)benzyl ester.

As used herein, the term “DMF” refers to N,N- dimethylformamide.

As used herein, the term “EDC” refers to 1-ethyl-3-(3-dimethylaminopropyl) carbodiimide.

As used herein, the language “effective amount” or “therapeutically effective amount” refers to a non-toxic but sufficient amount of the composition used in the practice of the invention that is effective to treat, prevent or ameliorate SARS-CoV-2 and/or SARS-CoV-1 infection in the body of a mammal. The desired treatment may be prophylactic and/or therapeutic. That result may be reduction and/or alleviation of the signs, symptoms, or causes of a disease or disorder, or any other desired alteration of a biological system. An appropriate therapeutic amount in any individual case may be determined by one of ordinary skill in the art using routine experimentation.

As used herein, the term “gp120 binder” refers to a small molecule, peptide or antibody that binds to the envelope protein gp120 of HIV-1.

As used herein, the term “HBTU” refers to O-benzotriazole-N,N,N′,N′-tetramethyl-uronium-hexafluoro-phosphate.

As used herein, the term “HEK” refers to Human embryonic kidney.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl. Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

As used herein, the term “medical intervention” means a set of one or more medical procedures or treatments that are required for ameliorating the effects of, delaying, halting or reversing a disease or disorder of a subject. A medical intervention may involve surgical procedures or not, depending on the disease or disorder in question. A medical intervention may be wholly or partially performed by a medical specialist, or may be wholly or partially performed by the subject himself or herself, if capable, under the supervision of a medical specialist or according to literature or protocols provided by the medical specialist.

As used herein, “natural amino acids” are represented by the full name thereof, by the three-letter code, as well as the one-letter code corresponding thereto, as indicated in the following table. The structure of amino acids and their abbreviations can also be found in the chemical literature, such as in Stryer, 1988, “Biochemistry”, 3^{rd} Ed., W. H. Freeman and Co., New York.

As used herein, the term “non-natural amino acid” corresponds to an amino acid that is not the L-isomer of one of the natural alpha-amino acids listed herein. Non-natural amino acids include, but are not limited to, the D-isomer of a natural amino acid, H₂N(CH₂CH₂O)ₙCH₂CH₂COOH (wherein MW varies from ~1000 Da to 10000 Da), H₂N(CH₂)ₙCOOH (wherein n is an integer that varies from 3 to 8), arginosuccinic acid, citrulline, cysteine sulfinic acid, 3,4-dihydroxy-phenylalanine, homocysteine, homoserine, ornithine, hydroxylysine, 4-hydroxy-proline, an N-Cbz-protected amino acid, 2,4-diaminobutyric acid, homoarginine, N-methyl-arginine, norleucine, N-methylaminobutyric acid, naphthylalanine, phenylglycine, beta-phenylproline, tert-leucine, 4-aminocyclohexyl-alanine, N-methyl-norleucine, 3,4-dehydroproline, N,N-dimethylaminoglycine, N-methylaminoglycine, 4-aminopiperidine-4-carboxylic acid, 6-aminocaproic acid (also known as Acp or 6-aminohexanoic acid), 6-aminocapramide (also known as AcpNH₂ or 6-aminohexanamide), beta-alanine (also known as bAla or βAla), bAlaNH₂ (or βAlaNH₂, and also known as 3-aminopropanamide), trans-4-(aminomethyl)-cyclohexanecarboxylic acid, 2-(aminomethyl)-benzoic acid, 3-(aminomethyl)-benzoic acid, 4-(aminomethyl)-benzoic acid, 1-aminocyclopentanecarboxylic acid, 1-aminocyclopropanecarboxylic acid, and 2-benzyl-5-aminopentanoic acid. Preferentially, the non-natural amino acid is selected from the group consisting of Acp, AcpNH₂, bAla and bAlaNH₂.

As used herein, the terms “peptide,” “polypeptide,” or “protein” are used interchangeably, and refer to a compound comprised of amino acid residues covalently linked by peptide bonds. A protein or peptide must contain at least two amino acids, and no limitation is placed on the maximum number of amino acids that can comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic has a N-terminus and a C-terminus. The N-terminus has an amino group, which may be free (i.e., as a NH₂ group) or appropriately protected (e.g., with a BOC or a Fmoc group). The C-terminus has a carboxylic group, which may be free (i.e., as a COOH group) or appropriately protected (e.g., as a benzyl or a methyl ester). A cyclic peptide does not necessarily have free N- or C-termini, since they are covalently bonded through an amide bond to form the cyclic structure.

As used herein, a “pharmaceutically acceptable carrier” means a pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; gar; buffering agents, such as magnesium hydroxide and aluminum hydroxide; alginic acid; pyrogen-free water; isotonic saline; Ringer’s solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, a “prophylactic” or “preventive” treatment is a treatment administered to a subject who does not exhibit signs of a disease or disorder or exhibits only early signs of the disease or disorder for the purpose of decreasing the risk of developing pathology associated with the disease or disorder.

As used herein, the term “PT” refers to peptide triazole.

As used herein, the term “SPR” refers to surface plasmon resonance.

As used herein, a “subject” or a “mammal” includes a human or a non-human mammal. Non-human mammals include, for example, livestock and pets, such as ovine, bovine, porcine, canine, feline and murine mammals. Preferably, the subject or mammal is human.

As used herein, the term “tBu” refers to tert-butyl.

As used herein, the term “TFA” refers to Trifluoroacetic acid.

As used herein, a “subject” or a “mammal” includes a human or a non-human mammal. Non-human mammals include, for example, livestock and pets, such as ovine, bovine, porcine, canine, feline and murine mammals. Preferably, the subject or mammal is human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less. The term “substantially free of” can mean having a trivial amount of, such that a composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

As used herein, a “therapeutic” treatment is a treatment administered to a subject who exhibits signs of pathology of a disease or disorder for the purpose of diminishing or eliminating those signs.

As used herein, the language “therapeutically effective amount” or “effective amount” refers to a non-toxic but sufficient amount of the composition used in the practice of the invention that is effective to treat, prevent or ameliorate HIV-1 infection in the body of a mammal. The desired treatment may be prophylactic and/or therapeutic. That result may be reduction and/or alleviation of the signs, symptoms, or causes of a disease or disorder, or any other desired alteration of a biological system. An appropriate therapeutic amount in any individual case may be determined by one of ordinary skill in the art using routine experimentation.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder.

As used herein, the term “viral envelope protein binder” refers to a small molecule, peptide or antibody that binds to at least one envelope protein of a virus.

### Compositions

### Lectin and/or Lectin DLIs

The present disclosure provides a composition for promoting virolysis and/or inhibition of infection of a virus in a mammal, the composition comprising a lectin mutant selected from the group consisting of mutant cyanovirin N (CVN) and mutant Griffithsin (GRFT).

In certain embodiments, the lectin mutant has at least 85% identity with CVN (SEQ ID NO:1) or GRFT (SEQ ID NO:2). In certain embodiments, the lectin mutant has at least 90% identity with CVN (SEQ ID NO:1) or GRFT (SEQ ID NO:2). In certain embodiments, the lectin mutant has at least 95% identity with CVN (SEQ ID NO:1) or GRFT (SEQ ID NO:2). In certain embodiments, the lectin mutant has at least 97.5% identity with CVN (SEQ ID NO:1) or GRFT (SEQ ID NO:2). In certain embodiments, the CVN mutant comprises a mutation at Pro51. In certain embodiments, the CVN mutant is P51G-CVN (SEQ ID NO:3).

In certain embodiments, the composition further comprises a S2 binding domain, wherein the lectin mutant and the binding domain are covalently linked by a flexible linker. In certain embodiments, the flexible linker comprises polyethylene glycol (PEG). In certain embodiments, the flexible linker comprises one or more amino acid residues. In certain embodiments, the linker comprises one to ten instances of GGGGS (SEQ ID NO:4). In certain embodiments, the linker comprises four instances of GGGGS (SEQ ID NO:4)

In certain embodiments, binding domain comprises a HIV-1 MPER or MPER-like Trp3 domain. In certain embodiments, the HIV-1 MPER or MPER-like Trp3 domain has at least 85% identity with HIV-1 MPER (SEQ ID NO:5) or MPER-like Trp3 domain (SEQ ID NO:6). In certain embodiments, the HIV-1 MPER or MPER-like Trp3 domain has at least 90% identity with HIV-1 MPER (SEQ ID NO:5) or MPER-like Trp3 domain (SEQ ID NO:6). In certain embodiments, the HIV-1 MPER or MPER-like Trp3 domain has at least 95% identity with HIV-1 MPER (SEQ ID NO:5) or MPER-like Trp3 domain (SEQ ID NO:6). In certain embodiments, the HIV-1 MPER or MPER-like Trp3 domain has at least 97.5% identity with HIV-1 MPER (SEQ ID NO:5) or MPER-like Trp3 domain (SEQ ID NO:6). In certain embodiments, CVN-L4-Trp3 comprises CVN linked to MPER-like Trp3 domain with 4 GGGGS (SEQ ID NO:4) units.

The present disclosure further provides a pharmaceutical composition comprising at least one composition of the present disclosure and at least one pharmaceutically acceptable carrier.

### Linkers

In certain embodiments, the compositions described herein comprise a linker. In certain embodiments, the covalent linkages may comprise amino acid and/or ethylene glycol units. In certain embodiments, the amino acid and/or ethylene glycol units are arranged in a linear manner. In certain embodiments, the linker is covalently linked at one terminus to the lectin and covalently linked at the opposite terminus to the binding domain. In certain embodiments, the covalent linkage comprises a carbon-heteroatom bond. In certain embodiments, the covalent linkage (i.e., covalent bond) comprises an ester, amide, ether, thioether, disubstituted amine, carbamate, carbonate ester, thioester, thiocarbamate, and dithiocarbamate. In certain embodiments, the covalent linkage comprises a carbon-carbon bond.

In certain embodiments, the linkers of the present disclosure may be directly conjugated to the lectin proteins described herein, for example, by direct covalent linkage to a backbone amino acid residue. In certain embodiments, the linker is covalently linked to the C-terminus of the lectin. In certain embodiments, the linker is covalently linked to the N-terminus of the lectin. In certain embodiments, the linker is covalently linked to a side-chain residue of the lectin.

In certain embodiments, the linkers of the present disclosure may be directly conjugated to the binding domain described herein, for example, by direct covalent linkage to a backbone amino acid residue of the binding domain. In certain embodiments, the linker is covalently linked to the C-terminus of the binding domain. In certain embodiments, the linker is covalently linked to the N-terminus of the binding domain. In certain embodiments, the linker is covalently linked to a side-chain residue of the binding domain.

### Cyclic Peptides

The present disclosure further provides a cyclic compound of formula (I), or a salt, solvate, enantiomer or diastereoisomer thereof:

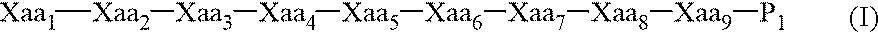

wherein in (I):
Xaa₁ is selected from the group consisting of absent, Glu and Arg;
Xaa₂ is selected from the group consisting of absent, Gly, Phe, Lys, Asp, Glu, Ile, Arg and Cit;
Xaa₃ is selected from the group consisting of absent, Asn, Asp, Ile, Glu and 2-cyclohexylglycine, wherein the alpha-amino group is optionally acylated with C₁-C₂₄ acyl;
Xaa₄ is selected from the group consisting of Asn, Asp, pyrazolyl-alanine, and thiazolyl-alanine;
Xaa₅ is a modified glycine of formula (III)

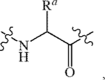

wherein R^{a} is selected from the group consisting of C₁-C₆ alkyl and C₃-C₆ cycloalkyl;
Xaa₆ is the modified proline of formula (IV)

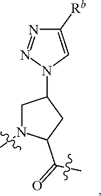

wherein R^{b} is phenyl substituted with R¹ or heteroaryl substituted with R¹, wherein R¹ is thienyl substituted with 1-2 C₁-C₆ alkyl groups;
Xaa₇ is selected from the group consisting of Trp and 3-(3-benzothienyl)-L-alanine;
Xaa₈ is selected from the group consisting of Ser, Thr, 2,4-diaminobutanoic acid, Orn and Lys;
Xaa₉ is selected from the group consisting of absent, 2,4-diaminobutanoic acid, Orn, Lys, Glu, Glu-Ala, Glu-Ala-Met, Glu-Ala-Met-Met, and 2-(2-(2-aminoethoxy)ethoxy)acetic acid;
P₁ is absent, or is a group that comprises at least one thiol group and is covalently linked through an amide bond to (i) the C-terminus of Xaa₉ if Xaa₉ is not absent, or (ii) the C-terminus of Xaa₈ if Xaa₉ is absent;
the side chain amino group of one residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉ forms an amide bond with the side chain carboxylic acid group of one residue selected from the group consisting of Glu at Xaa₂, Asp at Xaa₂, Glu at Xaa₃, Asp at Xaa₃ and Asp at Xaa₄; and
the C-terminus of Xaa₈ is optionally amidated if Xaa₉ and P₁ are absent, or the C-terminus of Xaa₉ is optionally amidated if P₁ is absent.

In certain embodiments, the cyclic compound is

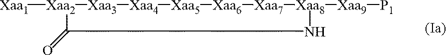

In certain embodiments, the cyclic compound is

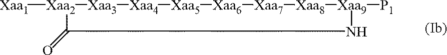

In certain embodiments, the cyclic compound is

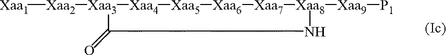

In certain embodiments, the cyclic compound is

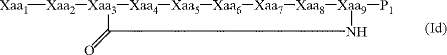

In certain embodiments, the cyclic compound is

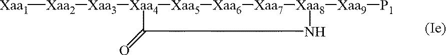

In certain embodiments, the cyclic compound is

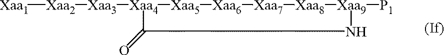

In certain embodiments, in (Ia)-(If), ‘NH’ is derived from the side chain amino group of a residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉, and ‘C═O’ is derived from the side chain carboxylic acid group of a residue selected from the group consisting of Glu at Xaa₂, Asp at Xaa₂, Glu at Xaa₃, Asp at Xaa₃, and Asp at Xaa₄.

In certain embodiments, the cyclic compound is a compound of formula (II):

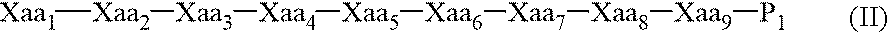

wherein in (II):
Xaa₁ is selected from the group consisting of absent, Glu and Arg;
Xaa₂ is selected from the group consisting of absent, Gly, Phe, Lys, Asp, Glu, Ile, Arg and Cit;
Xaa₃ is selected from the group consisting of Asn, Asp, and Glu, wherein the alpha-amino group of Xaa₃ is optionally acylated with C₁-C₂₄ acyl;
Xaa₄ is Asn, pyrazolyl-alanine, or thiazolyl-alanine;
Xaa₅ is Ile;
Xaa₆ is the modified proline of formula (IV)

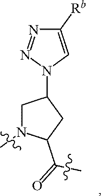

wherein R^{b} is phenyl substituted with R¹ or heteroaryl substituted with R¹, wherein R¹ is thienyl substituted with 1-2 C₁-C₆ alkyl groups;
Xaa₇ is Trp;
Xaa₈ is selected from the group consisting of Ser, Thr, 2,4-diaminobutanoic acid, Orn and Lys;
Xaa₉ is selected from the group consisting of absent, 2,4-diaminobutanoic acid, Orn, Lys, Glu, Glu-Ala, Glu-Ala-Met, Glu-Ala-Met-Met, and 2-(2-(2-aminoethoxy)ethoxy)acetic acid;
P₁ is absent, or is a group that comprises at least one thiol group and is covalently linked through an amide bond to (i) the C-terminus of Xaa₉ if Xaa₉ is not absent or (ii) the C-terminus of Xaa₈ if Xaa₉ is absent;
the side chain amino group of one residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉ forms an amide bond with the side chain carboxylic acid group of one residue selected from the group consisting of Glu at Xaa₂, Asp at Xaa₂, Glu at Xaa₃, and Asp at Xaa₃; and
the C-terminus of Xaa₈ is optionally amidated if Xaa₉ and P₁ are absent, or the C-terminus of Xaa₉ is optionally amidated if P₁ is absent.

In certain embodiments, R^{b} is phenyl substituted with 2-thienyl which is substituted with at least one C₁-C₆ alkyl group. In certain embodiments, R^{b} is phenyl substituted with 3-thienyl which is substituted with at least one C₁-C₆ alkyl group. In certain embodiments, the C₁-C₆ alkyl group is methyl. In certain embodiments, the C₁-C₆ alkyl group is ethyl. In certain embodiments, the C₁-C₆ alkyl group is propyl. In certain embodiments, the C₁-C₆ alkyl group is isopropyl. In certain embodiments, the C₁-C₆ alkyl group is butyl. In certain embodiments, the C₁-C₆ alkyl group is isobutyl. In certain embodiments, the C₁-C₆ alkyl group is n-butyl. In certain embodiments, the C₁-C₆ alkyl group is t-butyl.

In certain embodiments, Xaa₆ is

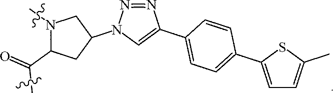

In certain embodiments, the cyclic compound is

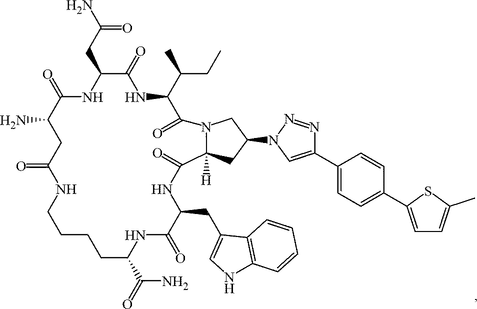

(also known as 29N2), (3S,6S,14S,17S,20S,24S,25aS)-3-((1H-indol-3-yl)methyl)-14-amino-17-(2-amino-2-oxoethyl)-20-((S)-sec-butyl)-24-(4-(4-(5-methylthiophen-2-yl)phenyl)-1H-1,2,3-triazol-1-yl)-1,4,12,15,18,21-hexaoxotetracosahydro-1H-pyrrolo[2,1-f][1,4,7,10,13,18]hexaazacyclotricosine-6-carboxamide, or a salt or solvate thereof. In certain embodiments, the cyclic compound is

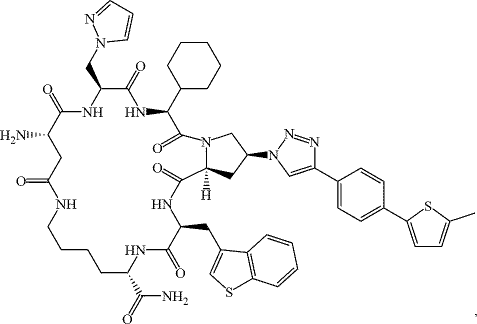

(3S,6S,14S,17S,20S,24S,25aS)-17-((1H-pyrazol-1-yl)methyl)-14-amino-3-(benzo[b]thiophen-3-ylmethyl)-20-cyclohexyl-24-(4-(4-(5-methylthiophen-2-yl)phenyl)-1H-1,2,3-triazol-1-yl)-1,4,12,15,18,21-hexaoxotetracosahydro-1H-pyrrolo[2,1-f][1,4,7,10,13,18]hexaazacyclotricosine-6-carboxamide, or a salt or solvate thereof. In certain embodiments, the cyclic compound is

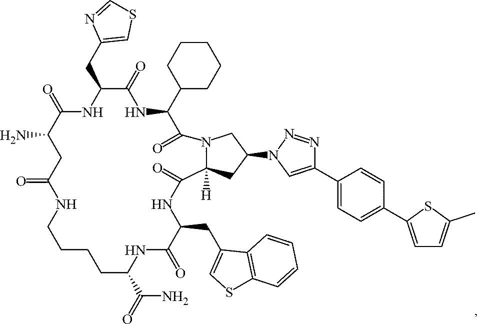

(3S,6S,14S,17S,20S,24S,25aS)-14-amino-3-(benzo[b]thiophen-3-ylmethyl)-20-cyclohexyl-24-(4-(4-(5-methylthiophen-2-yl)phenyl)-1H-1,2,3-triazol-1-yl)-1,4,12,15,18,21-hexaoxo-17-(thiazol-4-ylmethyl)tetracosahydro-1H-pyrrolo[2,1-f][1,4,7,10,13,18]hexaazacyclotricosine-6-carboxamide, or a salt or solvate thereof. In certain embodiments, the cyclic compound is

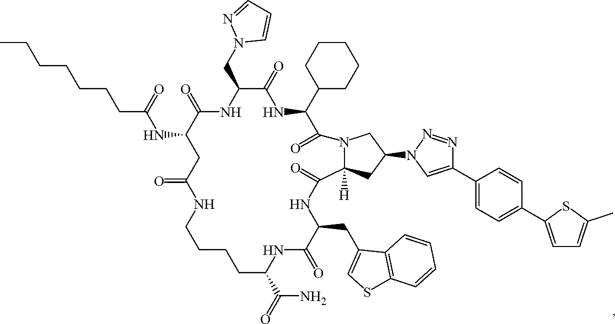

(3S,6S,14S,17S,20S,24S,25aS)-17-((1H-pyrazol-1-yl)methyl)-3-(benzo[b]thiophen-3-ylmethyl)-20-cyclohexyl-24-(4-(4-(5-methylthiophen-2-yl)phenyl)-1H-1,2,3-triazol-1-yl)-14-octanamido-1,4,12,15,18,21-hexaoxotetracosahydro-1H-pyrrolo[2,1-f][1,4,7,10,13,18]hexaazacyclotricosine-6-carboxamide, or a salt or solvate thereof. In certain embodiments, the cyclic compound is

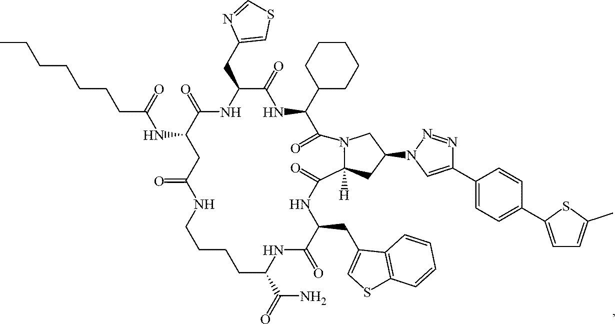

(3S,6S,14S,17S,20S,24S,25aS)-3-(benzo[b]thiophen-3-ylmethyl)-20-cyclohexyl-24-(4-(4-(5-methylthiophen-2-yl)phenyl)-1H-1,2,3-triazol-1-yl)-14-octanamido-1,4,12,15,18,21-hexaoxo-17-(thiazol-4-ylmethyl)tetracosahydro-1H-pyrrolo[2,1-f][1,4,7,10,13,18]hexaazacyclotricosine-6-carboxamide, or a salt or solvate thereof.

The present disclosure provides a pharmaceutical composition comprising at least one pharmaceutically acceptable carrier and at least one cyclic compound of any of the present disclosure.

In certain embodiments, the pharmaceutical composition further comprises at least one additional compound useful for treating viral infections.

In certain embodiments, the at least one additional compound is selected from the group consisting of antiviral combination drugs, entry and fusion inhibitors, integrase inhibitors, non-nucleoside reverse transcriptase inhibitors, nucleoside reverse transcriptase inhibitors, protease inhibitors, and any combinations thereof.

In certain embodiments, the compound is encapsulated in a hydrogel and/or liposome. In certain embodiments, the hydrogel and/or liposome is pH-responsive. In certain embodiments, the hydrogel comprises a polymerized mixture of methacrylic acid and PEG-monomethyl ether monomethacrylate.

In certain embodiments, the composition further comprises at least one pharmaceutically acceptable carrier. In other embodiments, the composition further comprises at least one additional compound useful for treating viral infections. In yet other embodiments, the at least one additional compound is selected from the group consisting of antiviral combination drugs, entry and fusion inhibitors, integrase inhibitors, non-nucleoside reverse transcriptase inhibitors, nucleoside reverse transcriptase inhibitors, protease inhibitors, and combinations thereof. In yet other embodiments, the peptide is encapsulated in a hydrogel and/or liposome. In yet other embodiments, the hydrogel and/or liposome is pH-responsive. In yet other embodiments, the hydrogel comprises a polymerized mixture of methacrylic acid and PEG-monomethyl ether monomethacrylate.

In certain embodiments, at least one compound of the invention is a component of a pharmaceutical composition further including at least one pharmaceutically acceptable carrier.

The compounds of the invention may possess one or more stereocenters, and each stereocenter may exist independently in either the (R)- or (S)-configuration. In certain embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/ or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention exist as tautomers. All tautomers are included within the scope of the compounds recited herein.

In certain embodiments, compounds described herein are prepared as prodrugs. A “prodrug” is an agent converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

### Salts

The compositions described herein may form salts with acids or bases, and such salts are included in the present invention. In certain embodiments, the salts are pharmaceutically acceptable salts. The term “salts” embraces addition salts of free acids or free bases that are compositions of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compositions of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compositions of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding composition by reacting, for example, the appropriate acid or base with the composition.

### Methods

The present disclosure further provides a method of treating, preventing, and/or ameliorating a viral infection in a subject, the method comprising administering to the subject a therapeutically effective amount of a composition comprising at least one composition of the present disclosure or a pharmaceutical composition of the present disclosure.

In certain embodiments, the viral infection is caused by a coronavirus. In certain embodiments, the coronavirus is selected from the group consisting of SARS-CoV-1 and SARS-CoV-2. In certain embodiments, the viral infection is caused by HIV-1.

The present disclosure further provides a method of promoting virolysis of a virus in a subject, the method comprising administering to the subject in need thereof a therapeutically effective amount of a composition comprising at least one composition of the present disclosure or a pharmaceutical composition of the present disclosure.

In certain embodiments, the virus is a coronavirus. In certain embodiments, the coronavirus is selected from the group consisting of SARS-CoV-1 and SARS-CoV-2. In certain embodiments, the virus is HIV-1.

In certain embodiments, the lectin is CVN (SEQ ID NO:1). In certain embodiments, the lectin is GRFT (SEQ ID NO:2). In certain embodiments, the lectin is P51G-CVN (SEQ ID NO:3). In certain embodiments, the composition comprises CVN-L4-Trp3. In certain embodiments, the subject is a mammal. In certain embodiments, the mammal is a human.

The present disclosure further provides a method of treating, preventing, and/or ameliorating HIV-1 infection in a subject in need thereof, the method comprising administering to the subject a therapeutically effective amount of at least one cyclic compound of the present disclosure or the pharmaceutical composition of the present disclosure.

In certain embodiments, the subject is further administered at least one additional compound useful for treating viral infections. In certain embodiments, the subject is a mammal. In certain embodiments, the mammal is a human.

### Combination Therapies

The compositions of the invention are useful in the methods of the invention in combination with one or more additional compounds useful for treating viral infections, such as but not limited to coronavirus (e.g., SARS-CoV-1 and/or SARS-CoV-2) and HIV infections. These additional compounds may comprise compounds or compositions identified herein, or compounds (e.g., commercially available compounds) known to treat, prevent, or reduce the symptoms of viral infections.

In non-limiting examples, the compositions of the invention may be used in combination with one or more of the following anti-HIV drugs:

HIV Combination Drugs: efavirenz, emtricitabine or tenofovir disoproxil fumarate (ATRIPLa®/BMS, Gilead); lamivudine or zidovudine (COMBIVIR®/GSK); abacavir or lamivudine (EPZICOM®/GSK); abacavir, lamivudine or zidovudine (TRIZIVIR®/GSK); emtricitabine, tenofovir disoproxil fumarate (TRUVADA®/Gilead).

Entry and Fusion Inhibitors: maraviroc (CELSENTRI®, SELZENTRY®/Pfizer); pentafuside or enfuvirtide (FUZEON®/Roche, Trimeris).

Integrase Inhibitors: raltegravir or MK-0518 (ISENTRESS®/Merck).

Non-Nucleoside Reverse Transcriptase Inhibitors: delavirdine mesylate or delavirdine (RESCRIPTOR®/Pfizer); nevirapine (VIRAMUNE®/Boehringer Ingelheim); stocrin or efavirenz (SUSTIVA®/BMS); etravirine (INTELENCE®/Tibotec).

Nucleoside Reverse Transcriptase Inhibitors: lamivudine or 3TC (EPIVIR®/GSK); FTC, emtricitabina or coviracil (EMTRIVA®/Gilead); abacavir (ZIAGEN®/GSK); zidovudina, ZDV, azidothymidine or AZT (RETROVIR®/GSK); ddI, dideoxyinosine or didanosine (VIDEX®/BMS); abacavir sulfate plus lamivudine (EPZICOM®/GSK); stavudine, d4T, or estavudina (ZERITO/BMS); tenofovir, PMPA prodrug, or tenofovir disoproxil fumarate (VIREAD®/Gilead).

Protease Inhibitors: amprenavir (AGENERASE®/GSK, Vertex); atazanavir (REYATAZ®/BMS); tipranavir (APTIVUS®/Boehringer Ingelheim); darunavir (PREZIST®/Tibotec); fosamprenavir (TELZIR®, LEXIVA®/GSK, Vertex); indinavir sulfate (CRIXIVAN®/Merck); saquinavir mesylate (INVIRASE®/Roche); lopinavir or ritonavir (KALETRA®/Abbott); nelfinavir mesylate (VIRACEPT®/Pfizer); ritonavir (NORVIR®/Abbott).

A synergistic effect may be calculated, for example, using suitable methods such as, for example, the Sigmoid-Eₘₐₓ equation (Holford & Scheiner, 19981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

Routes of administration of any of the compositions of the invention include oral, nasal, rectal, intravaginal, parenteral (e.g., IM, IV and SC), buccal, sublingual or topical. The regimen of administration may affect what constitutes an effective amount. The therapeutic formulations may be administered to the subject either prior to or after the onset of a viral infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat a viral infection in the subject. An effective amount of the therapeutic compound necessary to achieve a therapeutic effect may vary according to factors such as the state of the disease or disorder in the subject; the age, sex, and weight of the subject; and the ability of the therapeutic compound to treat a viral infection in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level depends upon a variety of factors, including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian may start doses of the compounds useful within the invention employed in the pharmaceutical composition at levels lower than that required in order to achieve the desired therapeutic effect and gradually increase the dosage until the desired effect is achieved.

In certain embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on the unique characteristics of the therapeutic compound and the particular therapeutic effect to be achieved, and the limitations inherent in the art of compounding/formulating such a therapeutic compound for the treatment of a SARS-CoV-2 and/or SARS-CoV-1 infection in a subject.

In certain embodiments, the compositions of the invention are formulated using one or more pharmaceutically acceptable excipients or carriers. In certain embodiments, the pharmaceutical compositions of the invention comprise a therapeutically effective amount of a compound useful within the invention and a pharmaceutically acceptable carrier.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In certain embodiments, the pharmaceutically acceptable carrier is not DMSO alone.

In certain embodiments, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any subject are determined by the attending physical taking all other factors about the subject into account.

Compounds useful within the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound useful within the invention is from about 1 mg and about 2,500 mg. In some embodiments, a dose of a compound useful within the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a SARS-CoV-2 antiviral) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments therebetween.

In certain embodiments, the present invention is directed to a packaged pharmaceutical composition comprising a container holding a therapeutically effective amount of a compound useful within the invention, alone or in combination with a second pharmaceutical agent; and instructions for using the compound to treat, prevent, or reduce one or more symptoms of a SARS-CoV-2 infection in a subject.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the invention, and a further layer providing for the immediate release of a medication for SARS-CoV-2 infection. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, In certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound’s ability to perform its intended function, e.g., treating, preventing, or reducing an SARS-CoV-2 infection in a subject.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, the compositions of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compositions of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pats. Nos. 6,340,475, 6,488,962, 6,451,808, 5,972,389, 5,582,837, and 5,007,790. Additional dosage forms of this invention also include dosage forms as described in U.S. Pat. Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention will depend on the age, sex and weight of the subject, the current medical condition of the subject and the nature of the infection by SARS-CoV-1, SARS-CoV-2, or HIV-1 being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Pseudovirus Infection Inhibition by Lectins

Recombinant SARS-CoV-1 and -2 pseudoviruses are produced by co-transfecting HEK-293T cells with the envelope-deficient pNL4-3d Env- Nanoluc+ and SARS-CoV spike protein plasmid (either SARS-CoV-1 or -2 envelope). The viruses are harvested after 48 hours of treatment and purified using a 6-20% iodixanol gradient before testing.

To validate the infectivity of the pseudoviruses produced and to determine the potency by which lectins and lectin-DLIs inhibit SARS-CoV-2 infection on ACE2 expressing cells, a single round luciferase reporter assay is used. HEK 293 ACE2 cells are seeded in a 96 well tissue culture plate prior to treating them with the purified pseudovirus in the presence of lectins. The extent of viral entry into the cells is measured by luminescence. To determine the ability of lectins to cause lytic inactivation of SARS-CoV-2, a p24 sandwich ELISA assay is used to quantify the amount of p24 released from virus particles. Furthermore, to confirm the irreversible nature of inactivation by lectins and lectin-DLIs, the viruses are preincubated with the inhibitors for various times (e.g., 0-24 hours). The viruses are purified via an iodixanol gradient before being tested for infectivity on HEK 293 ACE2 cells.

### Fully Infectious SARS-CoV-2 Virus Assay Methods

To determine whether lectins and/or lectin-DLIs block the ability of fully infectious SARS-CoV-2 to infect cells, fully infectious viruses were pretreated with a dose range of compound and virus (calculated to infect at various concentrations of the molecules of interest). Virus stocks are produced in Vero-E6 cells (kidney epithelial cells African green monkeys). The amount of virus inhibition is determined by standard plaque assay in human epithelial Calu-3 cell line to directly measure infectious virus production. Viral genomes are determined in parallel experiments by RT-PCR. The assays are repeated at least three times. Controls, included with each experiment, consist of untreated infected and uninfected cells. The concentrations yielding 50 and 90% inhibition of virus infection (IC₅₀ AND IC₉₀) are calculated. All experiments with fully infectious SARS-CoV-2 are performed in a BSL-3 laboratory.

### SPR Assay Configurations

To confirm CVN preference for S2, CVN binding to C-terminally captured S1, S2 and S monomer was measured. Preliminary data showed that the orientation of the CVN binding epitopes is crucial. The spike (S) presents under-processed oligomannose-type glycan clusters in the RBD, close to the ACE2 receptor binding motif, and on S2, consistent with the potential for CVN and MVN binding to S1 and S2. Binding assays are repeated on S1, S2, and S after specific mannosidase deglycosilation. specific, functional lectin binding residues are further localized using glycan-deleting point mutations by combining N-linked residue location with the trimeric prefusion conformation. For mutagenesis, the possible role of residues 234 and 709 were initially examined based on prior findings of the persistence of oligomannose in S1/S2 of SARS-CoV-1 and SARS-CoV-2.

### Interactions of Lectins And/or Lectin DLIs With SARS-CoV-2 Trimer Spike Protein

SARS-CoV proteins are captured on sensor surfaces from filtered, concentrated cell supernatants. SPR 4 flow-cell detection (B3000 and S200, Biacore), are used to measure CVN and other lectin interactions with the S1 and S2 domains, and with stabilized S-spike monomer and trimer. Orientation of ligands via C-terminal tags uses anti-Fc, 4-5His, and HA antibodies. SARS-CoV-2 and SARS-CoV-1 pseudoviruses use previously described, reversible cholesterol-PEG-biotin-tagging and capture to sensor coupled CaptAvidin according to the assay configurations proposed in FIG. 5. Sensor chips include CM5, CM3, the new PEG-coated sensor or C1. A second step called ‘capture coupling’ may be added to stabilize the captured ligands as required.

Specific mAb binding to gp120/gp41 in the membrane context and demonstrated the HIV envelope integrity in sCD4 binding (30 nM) to BaL.01 versus captured gp120 monomer. Dose-response plots of sCD4-Env were obtained with high reproducibility (SE±0.08, n=3) from capture to capture and detected a CD4 mimetic competition of sCD4 binding. Recombinant proteins (CVN, other lectins) are expressed in BL21(DE3) plysS (Promega) strains, glycosylated recombinant proteins are expressed in 293F in suspension. Recombinant pseudoviruses are obtained as described elsewhere herein.

Trimer in two formats allows assessment of interaction kinetics of CVN, as well as other lectins and DLIs, as described elsewhere herein. If affinity capture to SPR sensor surfaces is too low or unstable, alternative tags are incorporated. If antibodies for epitope detection on sensor-protein surfaces become limiting, academic and commercial sources for newly discovered antibody types are identified.

### Surface Plasmon Resonance (SPR) Assays

SPR experiments are performed on a Biacore 3000 optical biosensor (GE Healthcare). All experiments are carried out at 25° C. using standard PBS buffer pH=7.4 with 0.005% surfactant Tween and 2% DMSO.

Three flow cells in the CM5 chip were used for amine coupling of different ligands through standard 1-ethyl-3-(3-(dimethylamino)propyl) carbodiimide (EDC)/N-hydroxysuccinamide (NHS) chemistry. Flow cell 1 containing 2000 RUs of immobilized antibody 2B6R (α-human IL5R) served as a negative control for flow cells 2 and 3 each of which contained 2000 RUs of immobilized CD4 and 17b respectively.

For kinetic analyses, typically 2000-3000 RUsof protein reagents are immobilized on SPR chips, and analytes are passed over the surface at 50-100 µL/min. Surface regeneration is achieved by a 5 µL injection of 10 mM HCl solution at 100 µL/min. Analysis of peptide-mediated inhibition of gp120 binding to sCD4 and mAb 17b is achieved by injecting a fixed concentration of HIV-1YU-2 gp120 (250 nM), with increasing peptide concentrations, over sCD4 (2000 RU) and mAb 17b (1000 RU) surfaces for 5 minute association and 5 minute dissociation at a flow rate of 50 µl min-1 in PBS. Regeneration of the surface is achieved by a single 10 second pulse of 1.3 M NaCl / 35 mM NaOH and single 5-second pulse of 10 mM glycine, pH 1.5, for sCD4 and mAb 17b, respectively.

Data analysis of SPR competition data was performed using BIAevaluation v4.1.1 software (GE). To correct for nonspecific binding, response signals from buffer injection and from control flow cell were subtracted from all sensorgrams. Inhibition potencies were determined by calculating the inhibitor concentration required for 50% inhibition of maximal binding (IC₅₀). The inhibition curve was plotted and then fitted using the four-parameter equation as shown below using OriginPro 8 graphing software.

$$\text{Response =} {R}_{h i g h} - \frac{\left({R}_{h i g h} - {R}_{l o w}\right)}{1 + \left(\frac{\text{Cone}}{{\text{A}}_{\text{j}}}\right)} {\text{A}}_{2} \qquad \text{(1)}$$

where R_{high} is the response value at high inhibitor concentrations and R_{low} is response at low inhibitor concentrations. Conc. is the concentration of inhibitor, and A₁ and A₂ are fitting parameters. At the IC₅₀ the following is true:

$$\text{Response =} {R}_{h i g h} - \frac{\left({R}_{h i g h} - {R}_{l o w}\right)}{2} \qquad \text{(2)}$$

Under this condition, A₁ = Conc and is thus taken as the desired IC₅₀ parameter.

The cyclic peptides of the invention are tested against HIV-1 gp120 by SPR competition assays. A mixture of the cyclic peptide/gp120 solutions is passed over SPR chip immobilized with CD4 and 17b separately. The competition assay evaluates the ability of each peptide to inhibit the binding between gp120 and both soluble CD4 and 17b antibody, a surrogate for co-receptor.

### Recombinant DLIs

Methods for the recombinant production of CVN-DLIs in BL21(DE3) pLysS competent cells were established. Plasmids obtained in previous work for adapted for linker length, Trp3 sequence, and lectin type (Parajuli et al. Biochem. J. 2020, 475:931-957).

### Example 1: Irreversible Inactivation of SARS-CoV-1 and SARS-CoV-2 Pseudoviruses

Functional assays were conducted to determine the extent to which CVN and CVN-L4-Trp3 (CVN-DLI) can cause inhibition of SARS-CoV-2 infection of HEK 293 ACE2 cells and irreversible pseudovirus inactivation via cell-free virolysis. Both CVN and CVN-DLI inhibited infection at nanomolar potencies (FIG. 6A). Further, the lectin-DLI, CVN-L4-Trp3, was modestly more potent in inhibiting infection compared to CVN alone. No infection inhibition was observed for Bt-Alkyne-L3-Trp3, a biotinylated control containing the DLI Trp3 component on its own. Without wishing to be bound by theory, the small enhancement in infection inhibition potency observed with CVN-DLI may be a due to a dual-site interaction, a phenomenon previously observed with HIV-1.

In addition to measuring inhibition of infection, the ability of CVN and CVN-DLIs to cause irreversible inactivation of SARS-CoV-2 pseudoviruses was assessed. Both CVN and CVN-L4-Trp3 were capable of causing cell-free virolysis, while no effect was observed with Bt-Alkyne-L3-Trp3 (FIG. 6B). That CVN was potent virolytically with SARS-CoV-2 was unexpected.

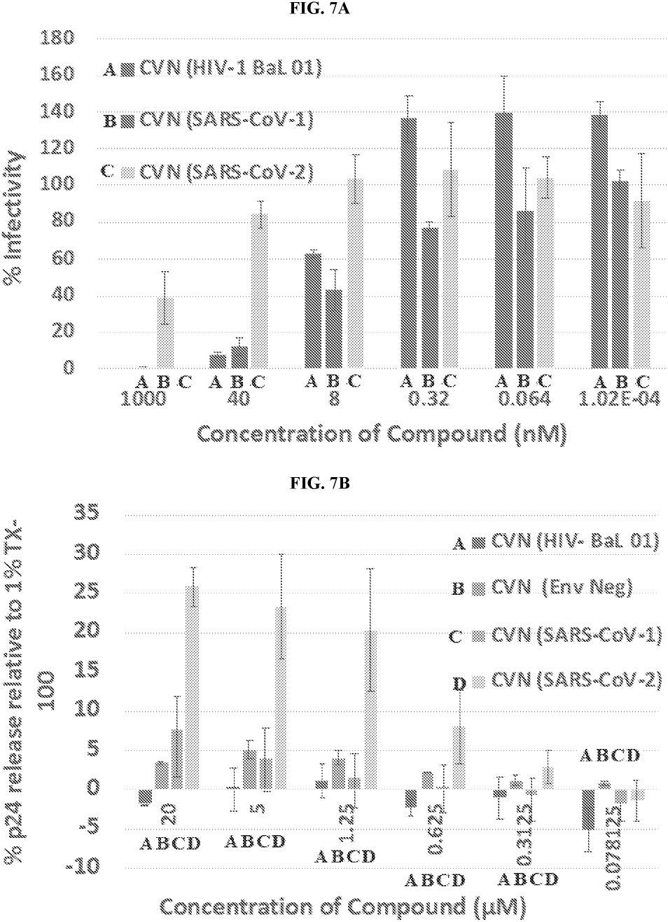

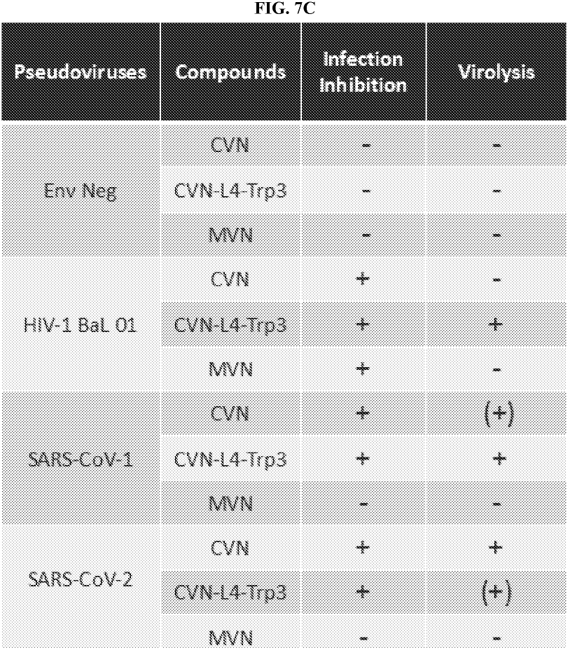

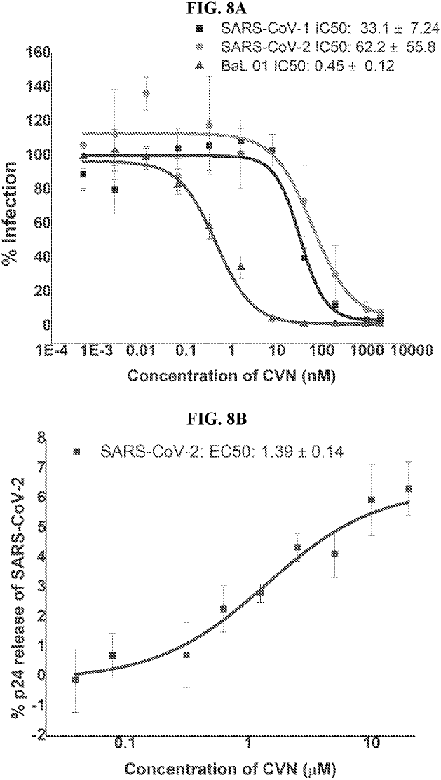

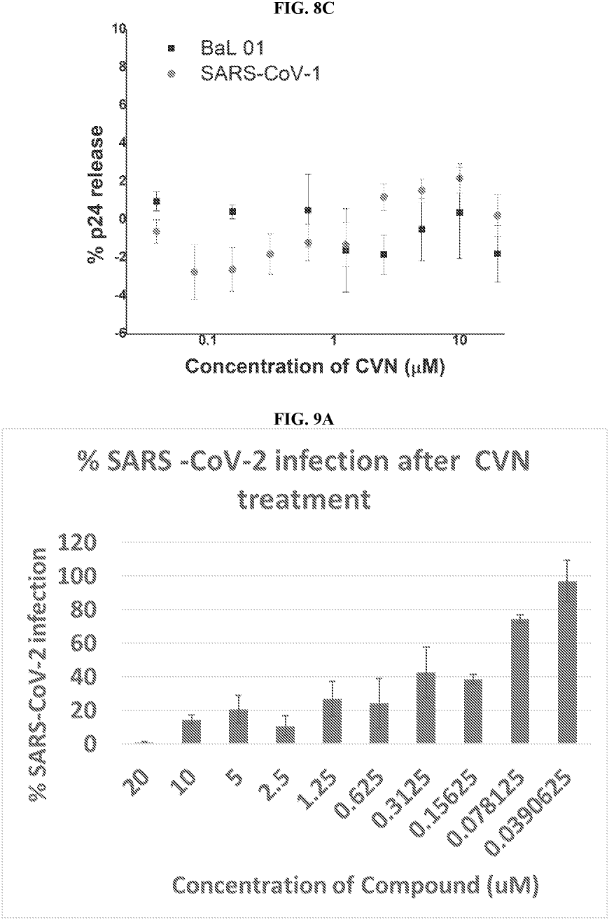

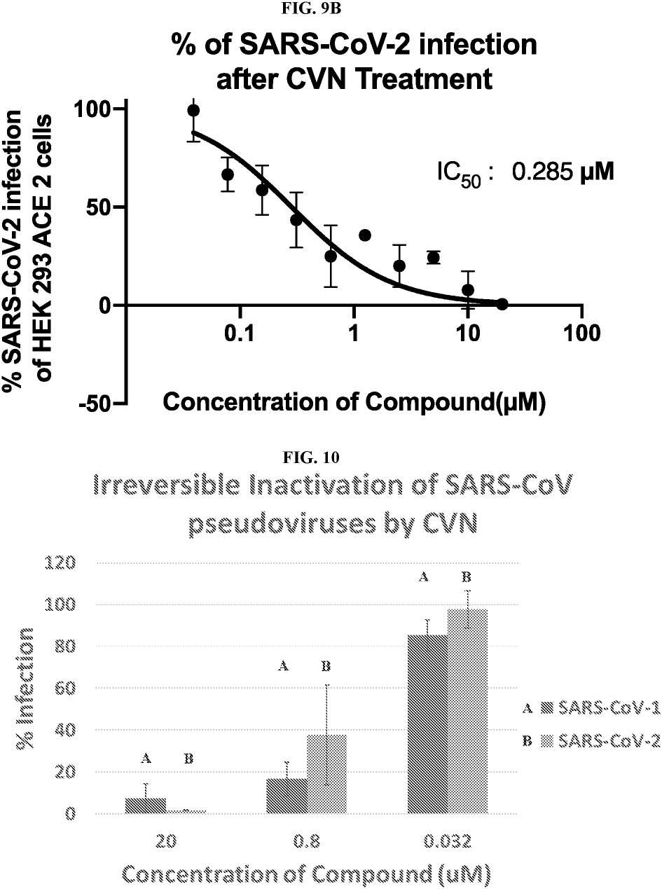

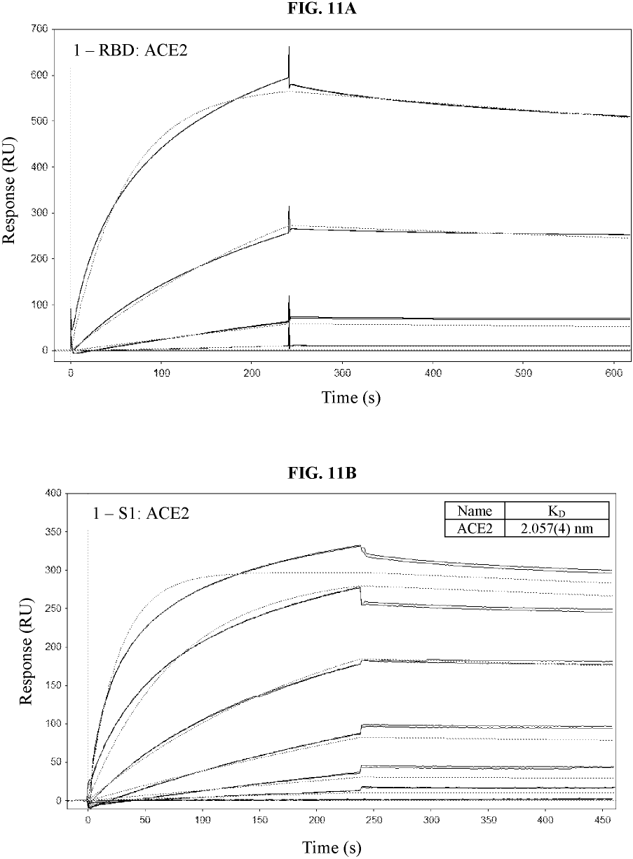

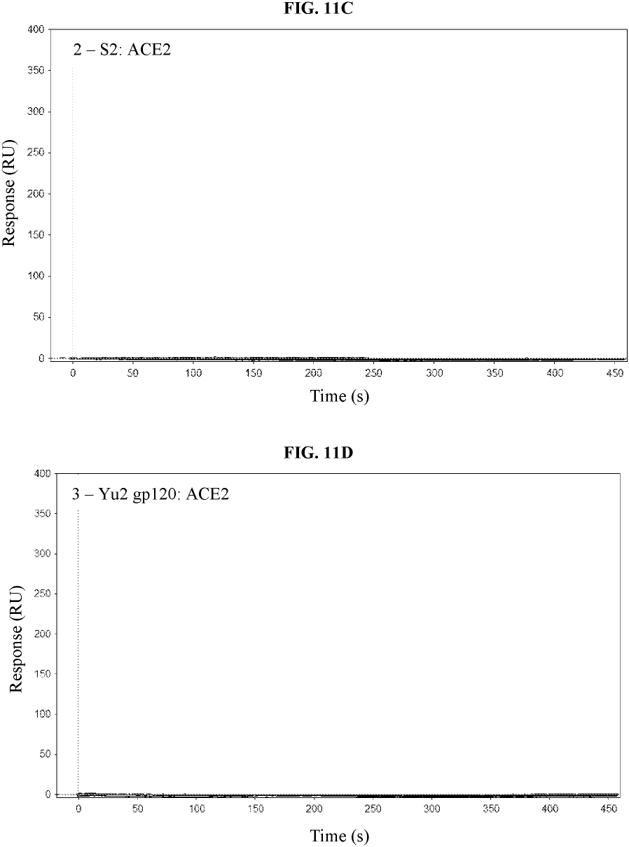

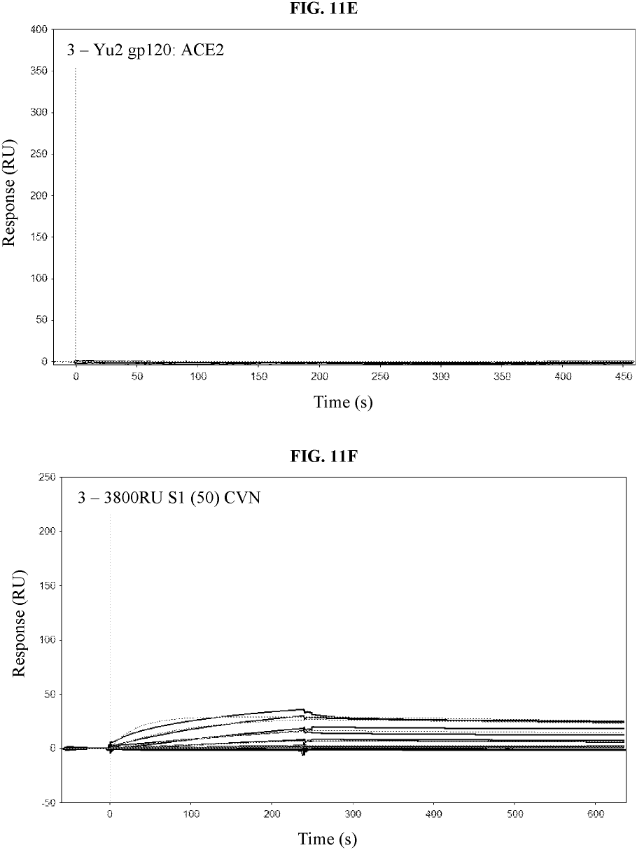

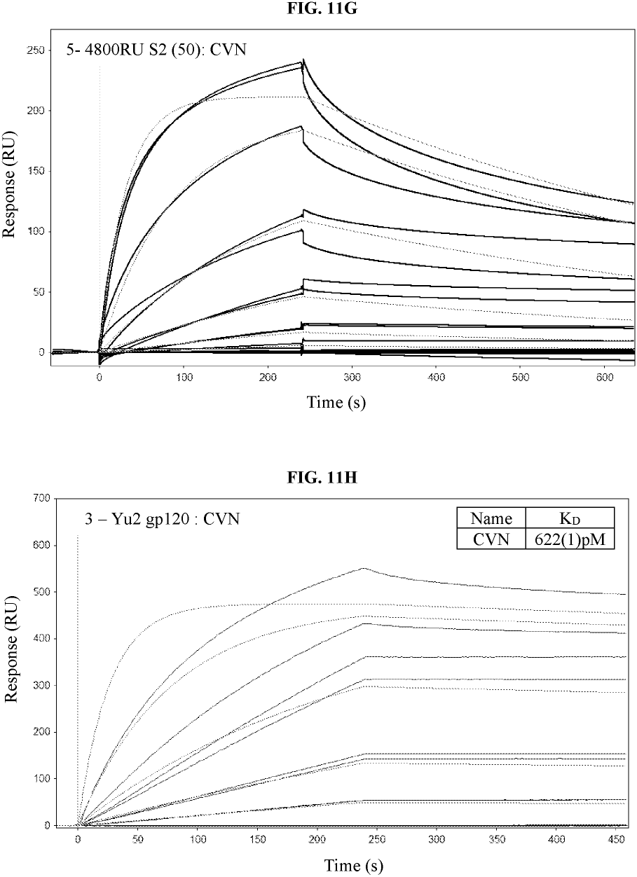

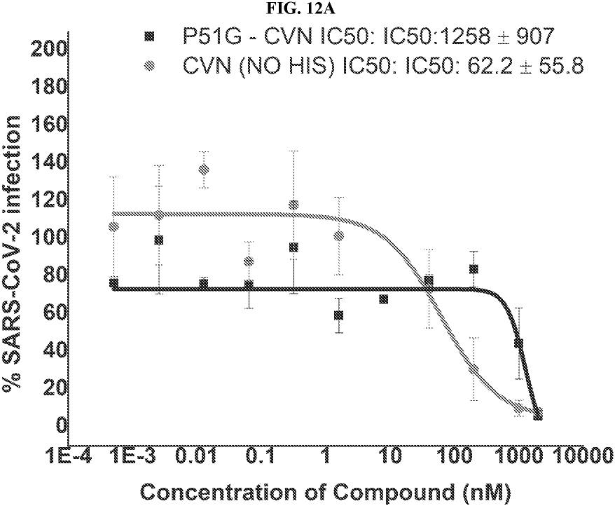

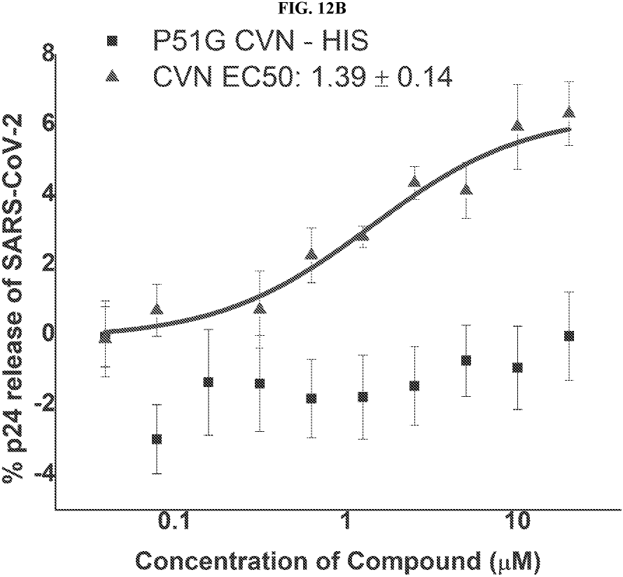

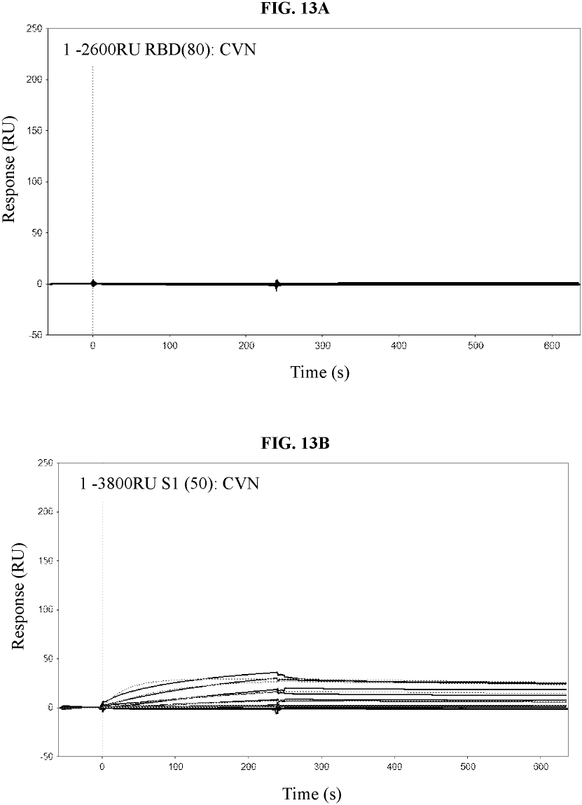

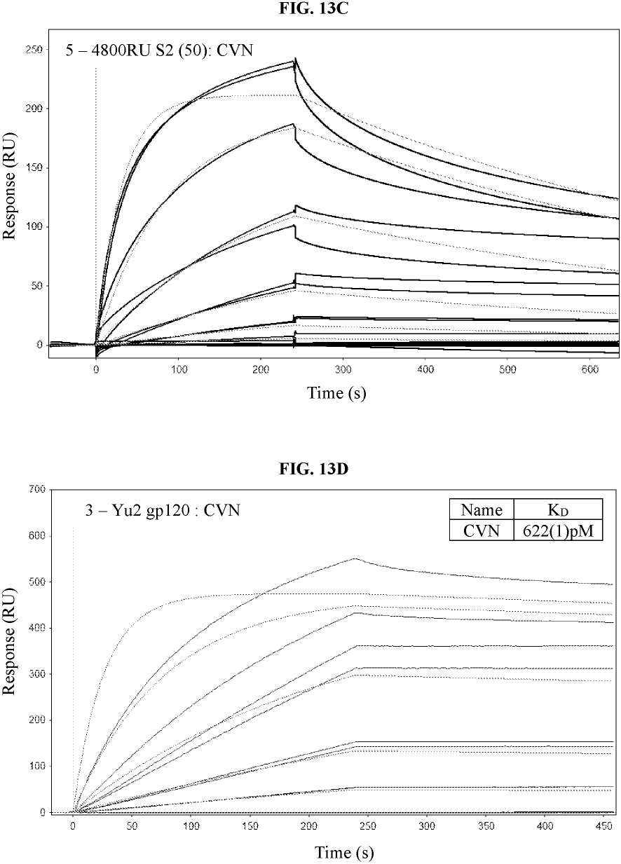

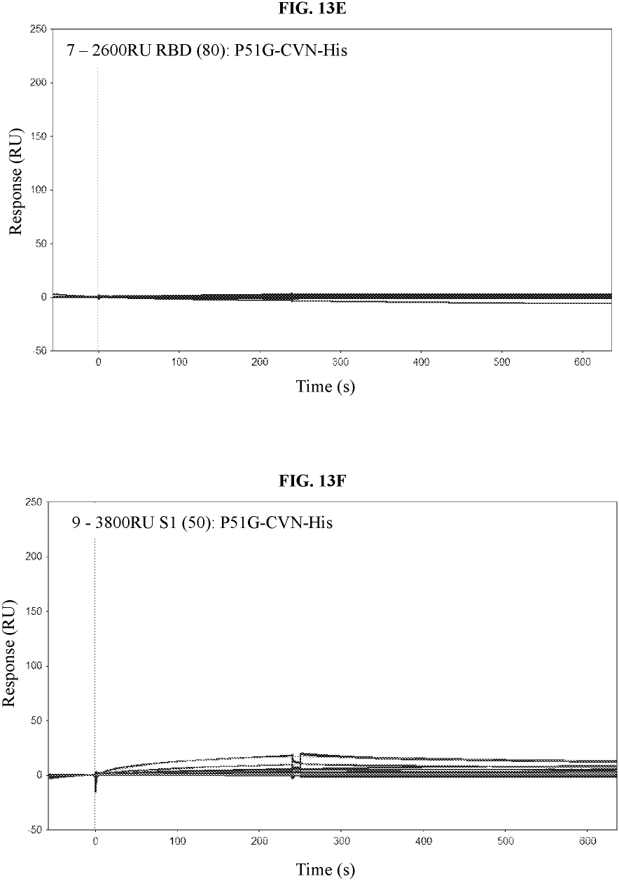

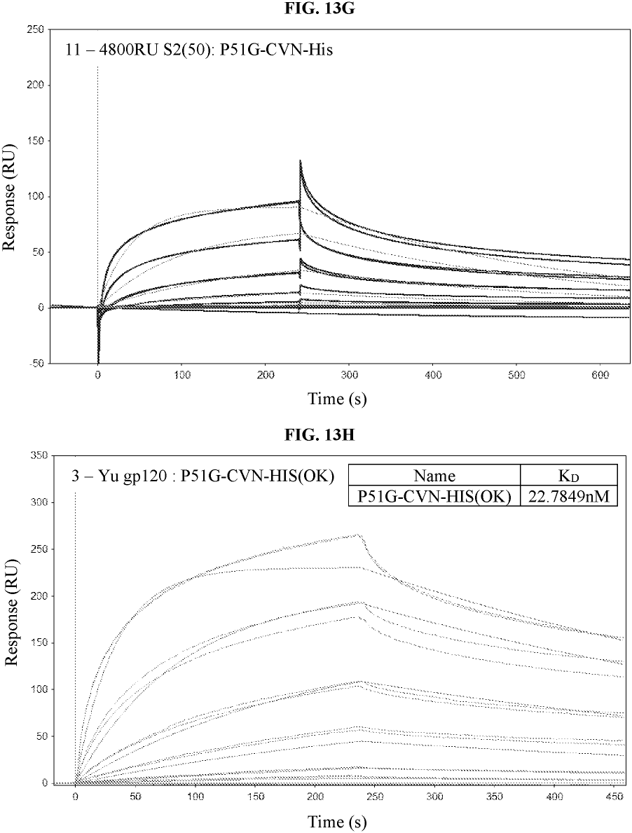

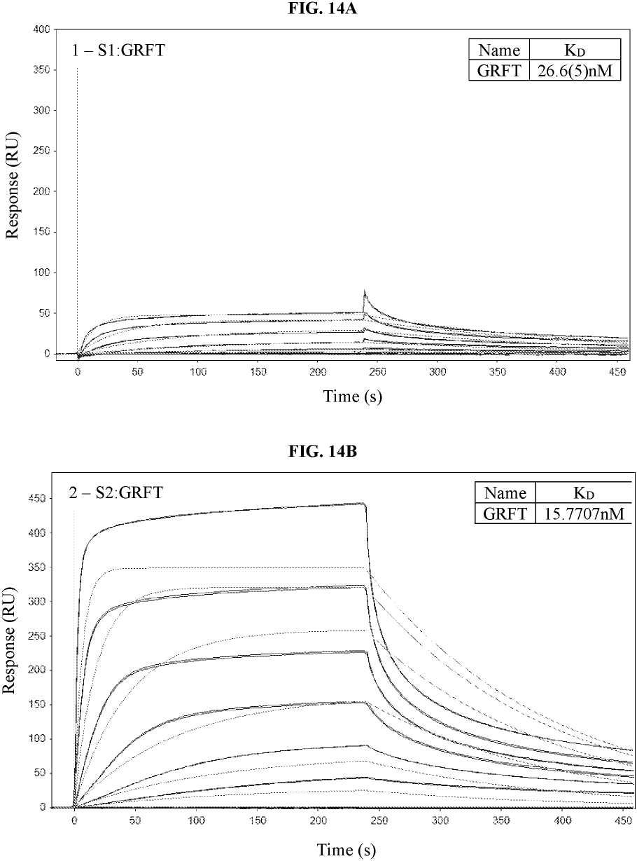

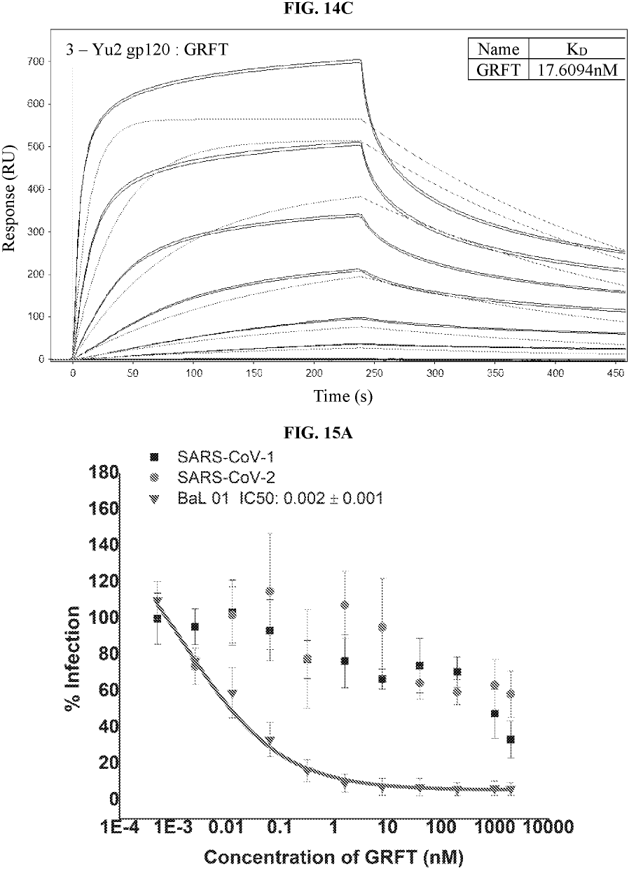

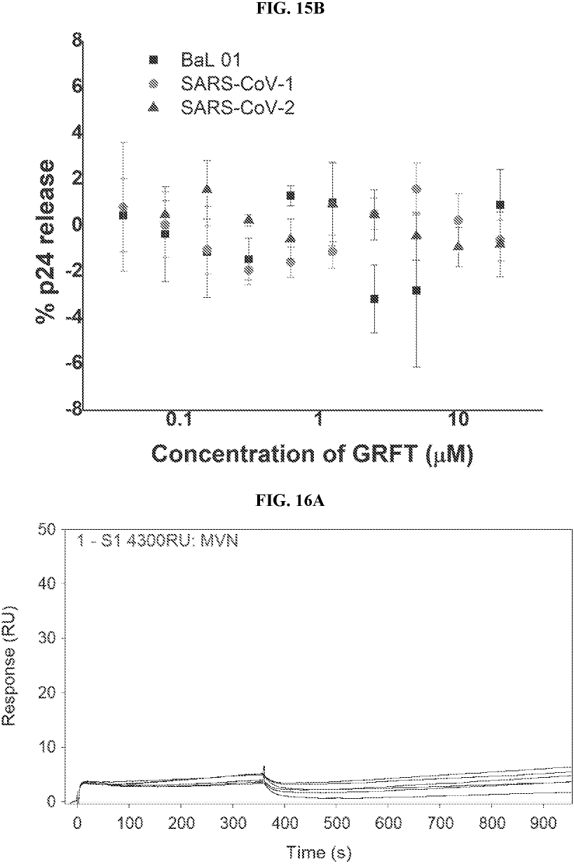

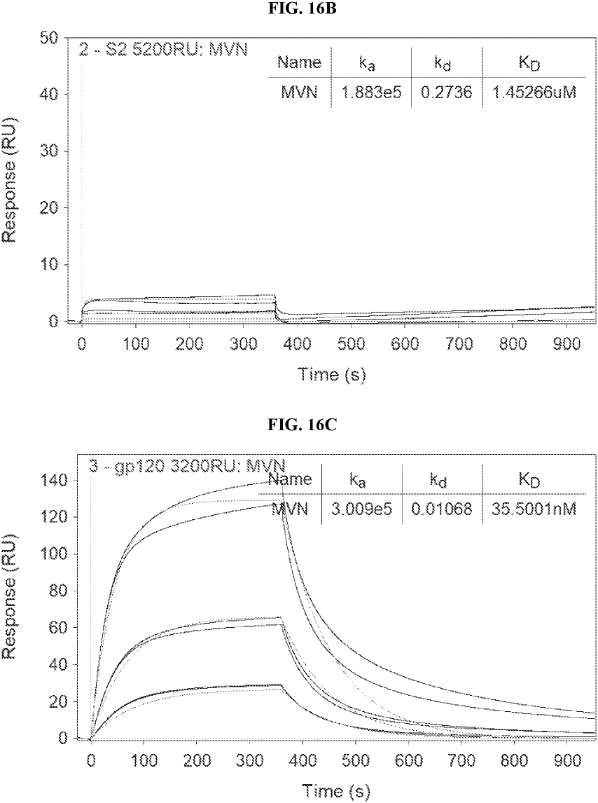

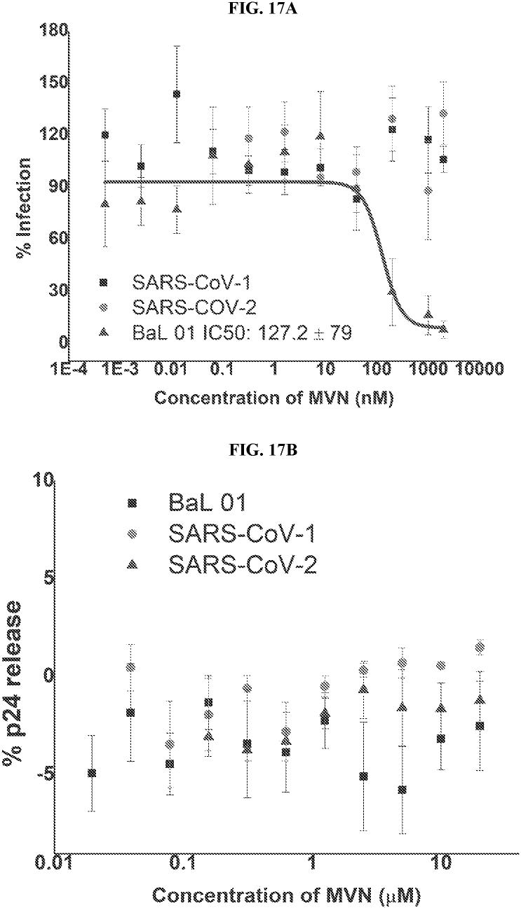

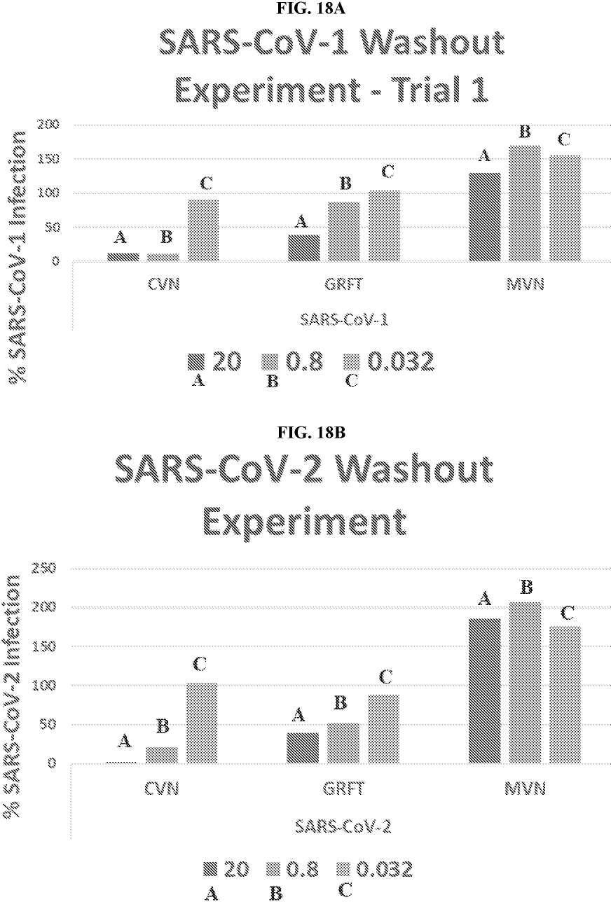

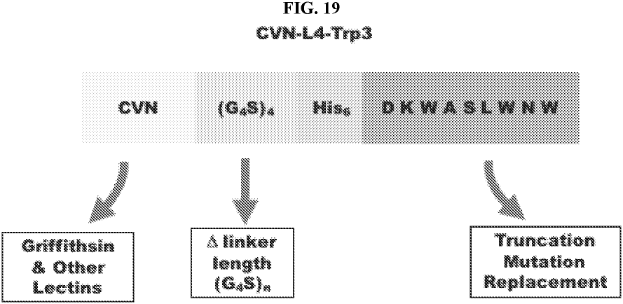

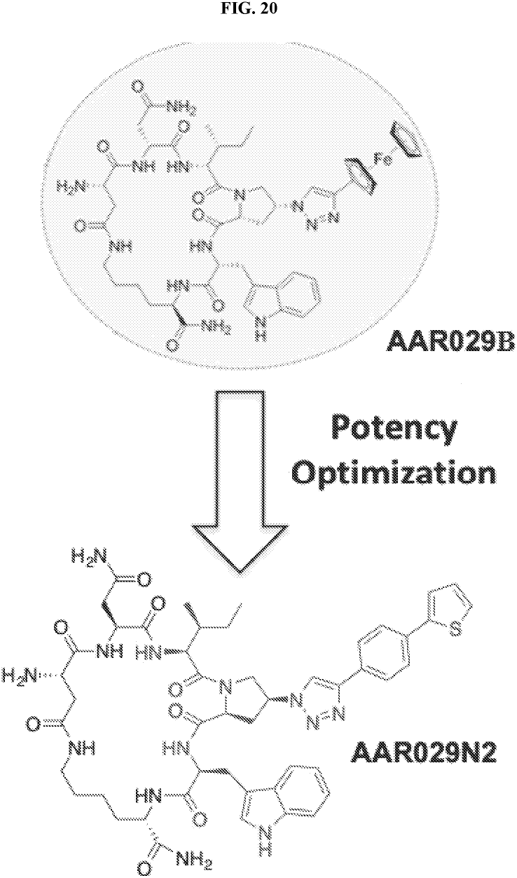

Control experiments were conducted with envelope negative (Env Neg), HIV-1, and SARS-CoV-1 pseudoviruses, wherein Env Neg is an envelope-deficient pseudovirus incapable of infecting HEK 293 ACE2 cells which served as a negative control (FIGS. 7A-7C).

In certain embodiments, CVN inhibits infection of HIV-1 in a subject. In certain embodiments, CVN inhibits infection of SARS-CoV-1 in a subject. In certain embodiments, CVN inhibits SARS-CoV-2 in a subject. In certain embodiments, CVN inhibits infection BaL01 in a subject. In certain embodiments, CVN causes improved virolysis of SARS-CoV-2 as compared to SARS-CoV-1 and BaL01, indicated by P24 release.

In certain embodiments, CVN is strongly lytic with SARS-CoV-2. In certain embodiments, little to no lysis is observed for CNV with Env-Neg or SARS-CoV-1. In certain embodiments, CVN demonstrated greater lytic activity for SARS-CoV-2 than CVN-L4-Trp3.

In certain embodiments, CVN binds to both the S1 and S2 subunits of the SARS-CoV pseudovirus. In certain embodiments, the stoichiometry of CVN binding to S2 is greater than observed for S1. In certain embodiments, no binding of CVN to the receptor binding domain (RBD) is observed.

Anti-SARS-CoV-2 functions correlate with glycan engagement. P51G-CVN, a mutant CVN wherein Pro 51 is mutated to Gly, prevents dimerization of CVN. In certain embodiments, P51G-CVN inhibits infection and/or virolysis of SARS-CoV-2. In certain embodiments, the inhibition of infection and/or virolysis of SARS-CoV-2 by P51G-CVN is reduced as compared to CVN.

In certain embodiments, P51G-CVN binds to both the S1 and S2 subunits of SARS-CoV pseudoviruses. In certain embodiments, P51G-CVN has reduced binding affinity for the S1 and S2 subunits of SARS-CoV pseudoviruses, as compared to CVN. In certain embodiments, the stoichiometry of CVN binding to S2 is greater than S1.

The inhibition of infection and/or virolysis of SARS-CoV pseudoviruses was further examined with additional lectins, including GRFT and MNV.

In certain embodiments, GRFT causes irreversible inactivation of SARS-CoV-2 and SARS-CoV-1, but not virolysis. In certain embodiments, the inhibition of infection observed with administration of GRFT is reduced as compared to CVN. In certain embodiments, GRFT binds to both the S1 and S2 subunits of SARS-CoV-2 pseudoviruses.

In certain embodiments, MNV does not cause irreversible inactivation of SARS-CoV-2 and SARS-CoV-1 pseudoviruses. No inhibition of infection or virolysis was observed with MVN. Additionally, low to minimal binding was observed of MVN to either the SARS-CoV S1 or S2 subunits.

Without wishing to be bound by theory, irreversible inactivation of virus, rather than just a non-covalent blockade, is possible upon glycan engagement. Irreversibility and p24 leakage results suggest that glycan engagement can facilitate disruption of the spike-membrane structure, perhaps due to spike conformational metastability.

Furthermore, differences in lectin potencies may reflect differences in glycan binding sites of the lectins, wherein CVN > GRFT >> MVN. In certain embodiments, CVN potency may rely on the 2-glycan site composition. Thus, glycans are potential targets for the development of nasal and/or oral treatments for SARS-CoV-2/COVID-19.

### Example 2: Lectin and Lectin-DLI Interaction Sites With SARS-CoV-2 Spike Complex

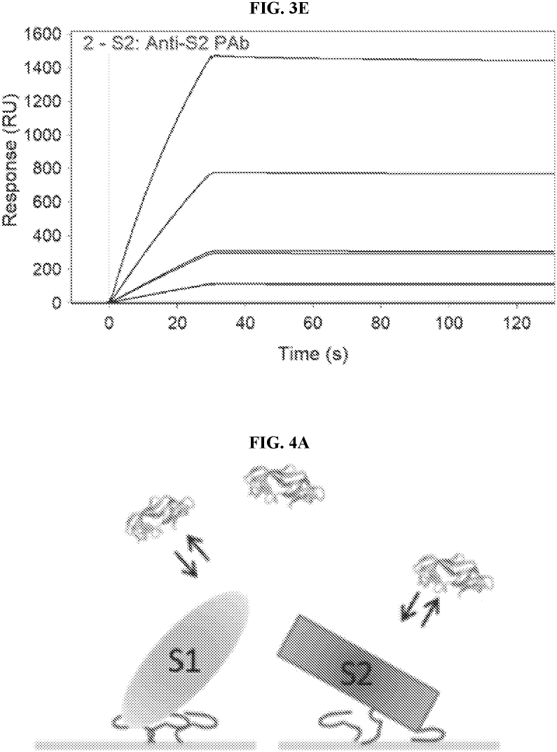

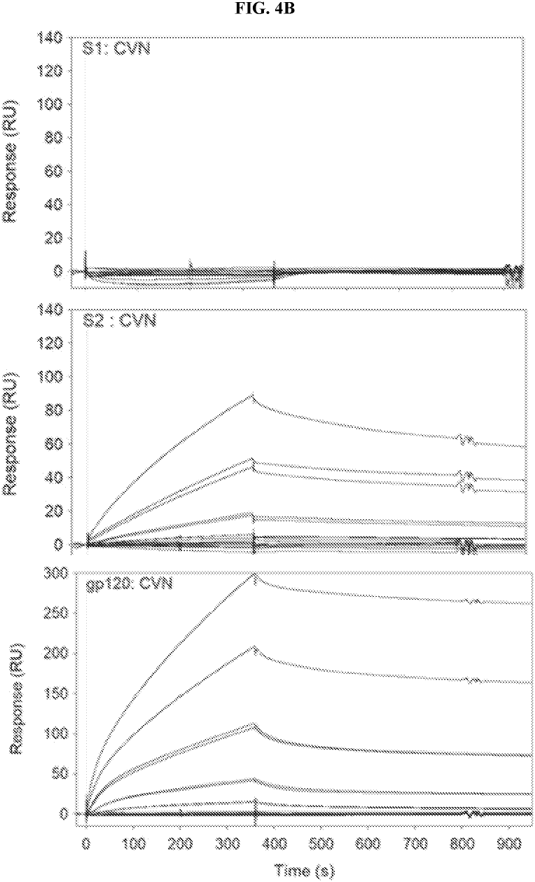

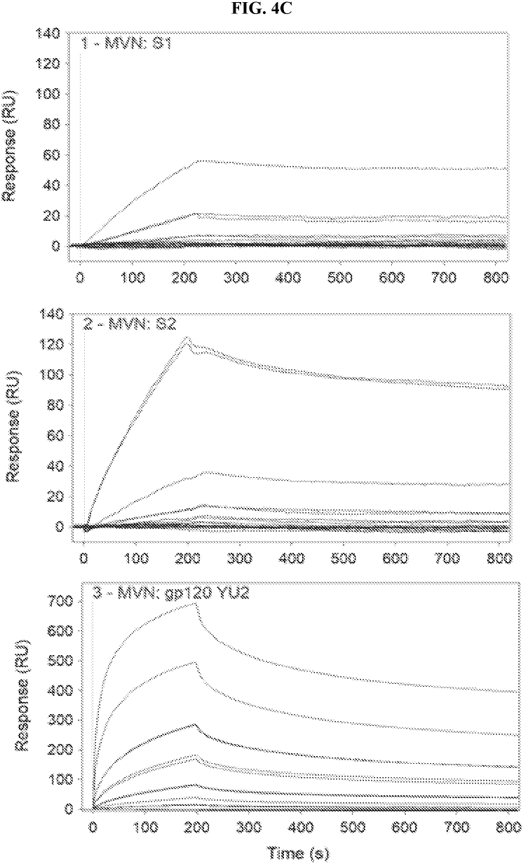

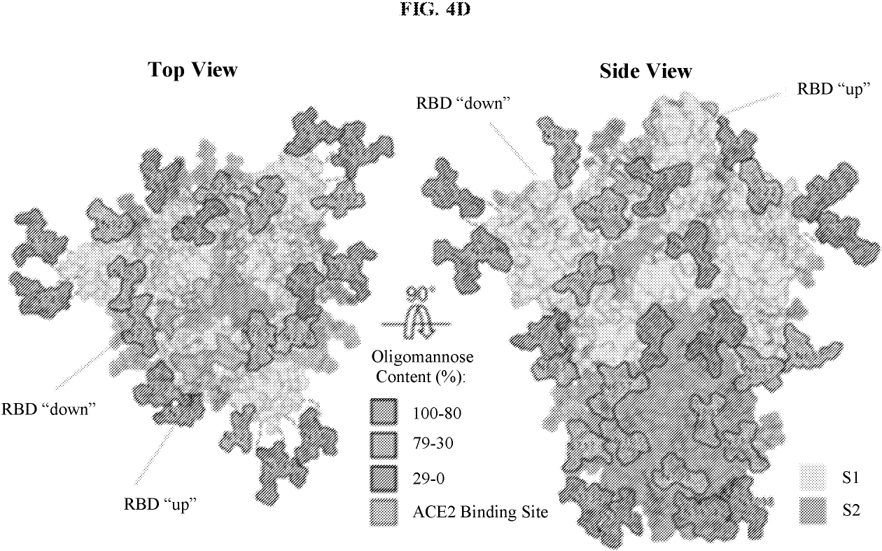

SARS-CoV-2 spike soluble components S1 and S2 were anchored on low dextran SPR sensor chips (FIG. 4A) and CVN injected at increasing concentrations. SPR 4 flow-cell detection (S200) of CVN binding to S1 and S2, with gp120 as a positive control, is shown in FIG. 4B. Specific binding to S2 but not to S1 was observed. Without wishing to be bound by theory, the preference of CVN for S2 may be related to the preponderance of high oligomannose glycans in S2 (FIG. 4C).

Non-lytic MVN (FIGS. 7A-7C) was also injected over the same surfaces and bound poorly to S2. Binding of both CVN and MVN to gp120 occurred as previously reported. In the opposite orientation, where the lectins are anchored to the surface, the CVN-S2 complexes were very stable compared to the MVN-S2 complexes. This stability cannot be explained by avidity as all CVN epitopes are equally accessible. Relatively lower binding of S1 to CVN and MVN was observed in this orientation. Overall, the SPR results so far reinforce the notion that the infection inhibition and lytic effects of CVN are driven by specific glycan engagement with S2.

### Example 3: Design of Modified Recombination Lectin DLIs

As a class I fusion protein, the SARS-CoV-2 spike undergoes major conformational rearrangements during entry and can be assumed to be metastable. The metastability of HIV-1 Env has been previously used to target the HIV-1 entry machine DLIs that impart stress to the Env spike by simultaneously binding to Env gp120 and what is reasoned to be the membrane-proximal external region (MPER) in gp41, with the dual-action mechanism causing viral poration. The S2 domain of SARS-CoV-2 spike not only binds CVN but also harbors a Trp-rich, MPER like region that may be targeted by the Trp3 or surrogate of DLIs.

As demonstrated herein, lectin-L4-Trp3 is lytically active with SARS-CoV-2 pseudovirus (FIGS. 6A-6B). However, the potency of the CVN-L4-Trp3 is less than that which is observed for lectin alone. Without wishing to be bound by theory, the lytic activity of the DLI with SARS-CoV-2 is, either predominately or in part, a consequence of the CVN domain. Further, the greater activity of CVN as compared to CVN-L4-Trp3 may reflect interfering engagement of the Trp3 domain.

SARS-CoV-2-specific lectin-DLIs can be derived with enhanced potency for the SARS-CoV-2 spike protein. The SARS-CoV-2 spike is more elongated spatially than the HIV-1 Env trimer, and it is possible that the encounter sites on the former for the lectin and Trp3 binding domains of the DLIs are differently spaced from each other than those in the latter. CVN binds preferentially to the more membrane-proximal spike subunit S2 in SARS-CoV-2 (FIGS. 4A-4D), in contrast to binding to the more membrane distal gp120 in HIV-1 Env.

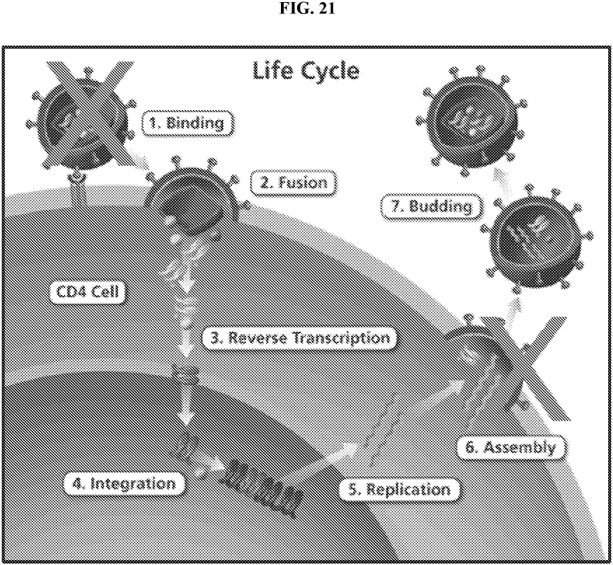

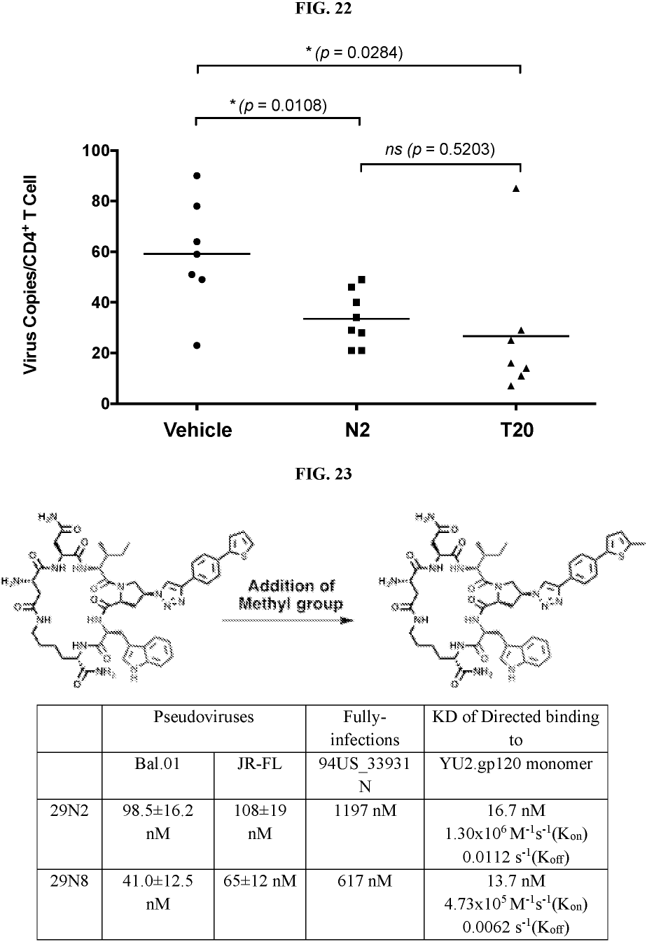

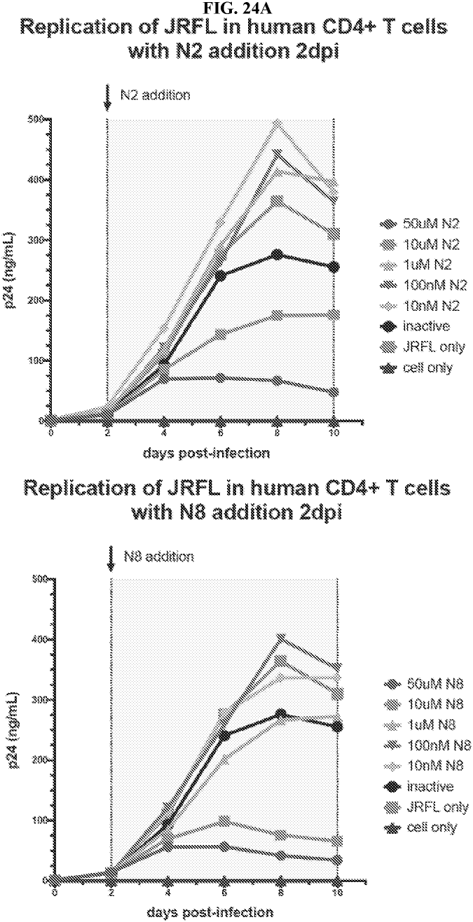

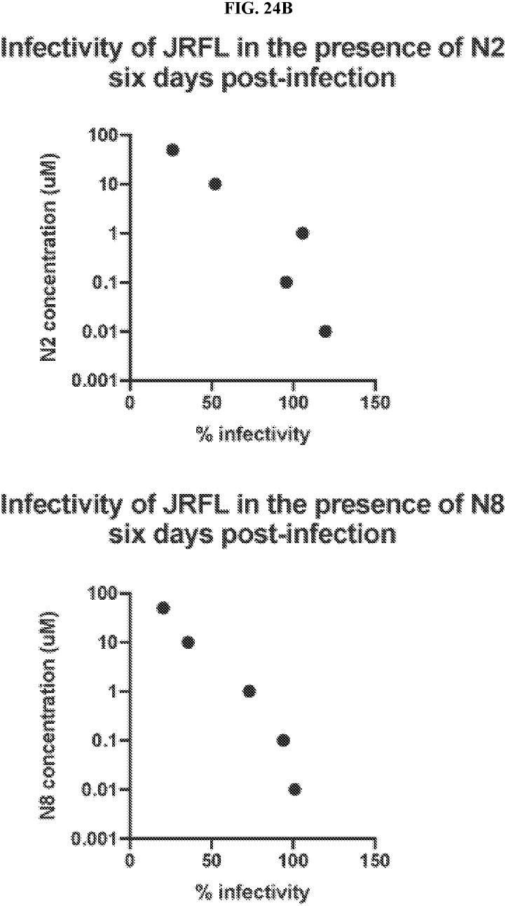

The impact of linker length variation has been examined (FIG. 19). In addition, modifications of the lectin and Trp3 domains themselves to optimize DLI inactivation of SARS-CoV-2 have been explored. Results indicate that GRFT, as CVN, binds to the S2 subunit, but not S1, of SARS-CoV-2.

In certain embodiments, the linker length may be varied to improve potency and/or activity of the lectin-DLI. In certain embodiments, the Trp3 domain of the lectin-DLI may be substituted for an alternative domain to improve potency and/or activity. In certain embodiments, the alternative domain comprises an alternative S2 site, including but not limited to T20-like sequences to target the S2 helical domain of the spike protein.

### Example 4: Cyclic Peptides

Cyclic peptides of the invention can be prepared using intramolecular cyclization according to the methods disclosed in International Application Publication No. WO 2016/094518 and International Application Publication No. WO 2018/053013, both of which are incorporated herein by reference in their entireties.

In certain embodiments, AAR029N8 (i.e., 29N8) showed improved HIV-1 inhibitory activity as compared to AAR029N2. In certain embodiments, the incorporation of an alkyl substituent on the thiophene group of AAR029N2 provides improved activity, bioavailability, and/or potency of the compound. In certain embodiments, the alkyl substituent is methyl.

### Sequence Listing

SEQ ID NO:1 Cyanovirin-N (CV-N)

Leu Gly Lys Phe Ser Gln Thr Cys Tyr Asn Ser Ala Ile Gln Gly
Ser Val Leu Thr Ser Thr Cys Glu Arg Thr Asn Gly Gly Tyr Asn
Thr Ser Ser Ile Asp Leu Asn Ser Val Ile Glu Asn Val Asp Gly
Ser Leu Lys Trp Gln Pro Ser Asn Phe Ile Glu Thr Cys Arg Asn
Thr Gln Leu Ala Gly Ser Ser Glu Leu Ala Ala Glu Cys Lys Thr
Arg Ala Gln Gln Phe Val Ser Thr Lys Ile Asn Leu Asp Asp His
Ile Ala Asn Ile Asp Gly Thr Leu Lys Tyr Glu

SEQ ID NO:2 GRFT

Ser Leu Thr His Arg Lys Phe Gly Gly Ser Gly Gly Ser Pro Phe
Ser Gly Leu Ser Ser Ile Ala Val Arg Ser Gly Ser Tyr Leu Asp
Xaa Ile Ile Ile Asp Gly Val His His Gly Gly Ser Gly Gly Asn
Leu Ser Pro Thr Phe Thr Phe Gly Ser Gly Glu Tyr Ile Ser Asn
Met Thr Ile Arg Ser Gly Asp Tyr Ile Asp Asn Ile Ser Phe Glu
Thr Asn Met Gly Arg Arg Phe Gly Pro Tyr Gly Gly Ser Gly Gly
Ser Ala Asn Thr Leu Ser Asn Val Lys Val Ile Gln Ile Asn Gly
Ser Ala Gly Asp Tyr Leu Asp Ser Leu Asp Ile Tyr Tyr Glu Gln
Tyr

SEQ ID NO:3 P51G-CVN

Leu Gly Lys Phe Ser Gln Thr Cys Tyr Asn Ser Ala Ile Gln Gly
Ser Val Leu Thr Ser Thr Cys Glu Arg Thr Asn Gly Gly Tyr Asn
Thr Ser Ser Ile Asp Leu Asn Ser Val Ile Glu Asn Val Asp Gly
Ser Leu Lys Trp Gln Gly Ser Asn Phe Ile Glu Thr Cys Arg Asn
Thr Gln Leu Ala Gly Ser Ser Glu Leu Ala Ala Glu Cys Lys Thr
Arg Ala Gln Gln Phe Val Ser Thr Lys Ile Asn Leu Asp Asp His
Ile Ala Asn Ile Asp Gly Thr Leu Lys Tyr Glu

SEQ ID NO:4 Linker 1

Gly Gly Gly Gly Ser

SEQ ID NO:5 HIV-1 MPER

Asp Lys Trp Ala Ser Leu Trp Asn Trp Phe Asn Ile Thr Asn Trp
Leu Trp Tyr Ile Lys

SEQ ID NO:6 MPER-like TRP3

Asp Lys Trp Ala Ser Leu Trp Asn Trp

### Enumerated Embodiments

The following exemplary embodiments are provided, the numbering of which is not to be construed as designating levels of importance:

Embodiment 1 provides a composition for promoting virolysis and/or inhibition of infection of a virus in a mammal, the composition comprising a lectin mutant selected from the group consisting of mutant cyanovirin N (CVN) and mutant Griffithsin (GRFT).

Embodiment 2 provides the composition of Embodiment 1, wherein the lectin mutant has at least 85% identity with CVN (SEQ ID NO:1) or GRFT (SEQ ID NO:2).

Embodiment 3 provides the composition of Embodiment 1 or 2, wherein the lectin mutant has at least 90% identity with CVN (SEQ ID NO:1) or GRFT (SEQ ID NO:2).

Embodiment 4 provides the composition of any of Embodiments 1-3, wherein the lectin mutant has at least 95% identity with CVN (SEQ ID NO:1) or GRFT (SEQ ID NO:2).

Embodiment 5 provides the composition of any of Embodiments 1-4, wherein the lectin mutant has at least 97.5% identity with CVN (SEQ ID NO:1) or GRFT (SEQ ID NO:2).

Embodiment 6 provides the composition of any of Embodiments 1-5, wherein the CVN mutant comprises a mutation at Pro51, optionally wherein the CVN mutant is P51G-CVN (SEQ ID NO:3).

Embodiment 7 provides the composition of any of Embodiments 1-6, further comprising a S2 binding domain, wherein the lectin mutant and the binding domain are covalently linked by a flexible linker.

Embodiment 8 provides the composition of Embodiment 7, wherein the flexible linker comprises polyethylene glycol (PEG).

Embodiment 9 provides the composition of Embodiment 7 or 8, wherein the flexible linker comprises one or more amino acid residues.

Embodiment 10 provides the composition of Embodiment 9, wherein the linker comprises one to ten instances of GGGGS (SEQ ID NO:4).

Embodiment 11 provides the composition of Embodiment 10, wherein the linker comprises four instances of GGGGS (SEQ ID NO:4)

Embodiment 12 provides the composition of any of Embodiments 7-11, wherein the binding domain comprises a HIV-1 MPER or MPER-like Trp3 domain.

Embodiment 13 provides the composition of Embodiment 12, wherein the HIV-1 MPER or MPER-like Trp3 domain has at least 85% identity with HIV-1 MPER (SEQ ID NO:5) or MPER-like Trp3 domain (SEQ ID NO:6).

Embodiment 14 provides a pharmaceutical composition comprising at least one composition of any of Embodiments 1-13 and at least one pharmaceutically acceptable carrier.

Embodiment 15 provides a method of treating, preventing, and/or ameliorating a viral infection in a subject, the method comprising administering to the subject a therapeutically effective amount of a composition comprising at least one composition of any of Embodiments 1-13 or the pharmaceutical composition of Embodiment 14.

Embodiment 16 provides the method of Embodiment 15, wherein the viral infection is caused by a coronavirus.

Embodiment 17 provides the method of Embodiment 16, wherein the coronavirus is selected from the group consisting of SARS-CoV-1 and SARS-CoV-2.

Embodiment 18 provides the method of Embodiment 15, wherein the viral infection is caused by HIV-1.

Embodiment 19 provides a method of promoting virolysis of a virus in a subject, the method comprising administering to the subject in need thereof a therapeutically effective amount of a composition comprising at least one composition of any of Embodiments 1-13 or the pharmaceutical composition of Embodiment 14.

Embodiment 20 provides the method of Embodiment 19, wherein the virus is a coronavirus.

Embodiment 21 provides the method of Embodiment 20, wherein the coronavirus is selected from the group consisting of SARS-CoV-1 and SARS-CoV-2.

Embodiment 22 provides the method of Embodiment 19, wherein the virus is HIV-1.

Embodiment 23 provides the method of any of Embodiments 15-22, wherein the lectin is CVN (SEQ ID NO:1).

Embodiment 24 provides the method of any of Embodiments 15-22, wherein the lectin is GRFT (SEQ ID NO:2).

Embodiment 25 provides the method of any of Embodiments 15-22, wherein the lectin is P51G-CVN (SEQ ID NO:3).

Embodiment 26 provides the method of any of Embodiments 15-22, wherein the composition comprises CVN-L4-Trp3.

Embodiment 27 provides the method of any of Embodiments 15-26, wherein the subject is a mammal.

Embodiment 28 provides the method of Embodiment 27, wherein the mammal is a human.

Embodiment 29 provides a cyclic compound of formula (I), or a salt, solvate, enantiomer or diastereoisomer thereof:

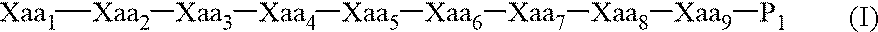

wherein in (I):
Xaa₁ is selected from the group consisting of absent, Glu and Arg;
Xaa₂ is selected from the group consisting of absent, Gly, Phe, Lys, Asp, Glu, Ile, Arg and Cit;
Xaa₃ is selected from the group consisting of absent, Asn, Asp, Ile, Glu and 2-cyclohexylglycine, wherein the alpha-amino group is optionally acylated with C₁-C₂₄ acyl;
Xaa₄ is selected from the group consisting of Asn, Asp, pyrazolyl-alanine, and thiazolyl-alanine;
Xaa₅ is a modified glycine of formula (III)

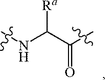

wherein R^{a} is selected from the group consisting of C₁-C₆ alkyl and C₃-C₆ cycloalkyl;
Xaa₆ is the modified proline of formula (IV)

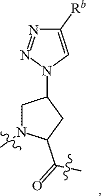

wherein R^{b} is phenyl substituted with R¹ or heteroaryl substituted with R¹, wherein R¹ is thienyl substituted with 1-2 C₁-C₆ alkyl groups;
Xaa₇ is selected from the group consisting of Trp and 3-(3-benzothienyl)-L-alanine;
Xaa₈ is selected from the group consisting of Ser, Thr, 2,4-diaminobutanoic acid, Orn and Lys;
Xaa₉ is selected from the group consisting of absent, 2,4-diaminobutanoic acid, Orn, Lys, Glu, Glu-Ala, Glu-Ala-Met, Glu-Ala-Met-Met, and 2-(2-(2-aminoethoxy)ethoxy)acetic acid;
P₁ is absent, or is a group that comprises at least one thiol group and is covalently linked through an amide bond to (i) the C-terminus of Xaa₉ if Xaa₉ is not absent, or (ii) the C-terminus of Xaa₈ if Xaa₉ is absent;
the side chain amino group of one residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉ forms an amide bond with the side chain carboxylic acid group of one residue selected from the group consisting of Glu at Xaa₂, Asp at Xaa₂, Glu at Xaa₃, Asp at Xaa₃ and Asp at Xaa₄; and
the C-terminus of Xaa₈ is optionally amidated if Xaa₉ and P₁ are absent, or the C-terminus of Xaa₉ is optionally amidated if P₁ is absent.

Embodiment 30 provides the cyclic compound of Embodiment 29, which is selected from the group consisting of:

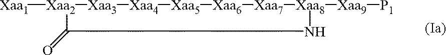

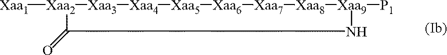

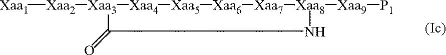

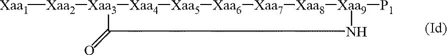

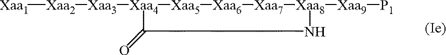

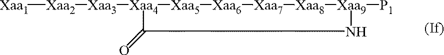

wherein in (Ia)-(If):
‘NH’ is derived from the side chain amino group of a residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉, and
‘C═O’ is derived from the side chain carboxylic acid group of a residue selected from the group consisting of Glu at Xaa₂, Asp at Xaa₂, Glu at Xaa₃, Asp at Xaa₃, and Asp at Xaa₄.

Embodiment 31 provides the cyclic compound of Embodiment 29, which is the cyclic compound of formula (II):

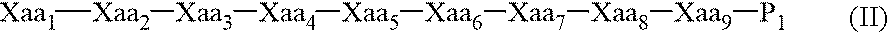

wherein in (II):
Xaa₁ is selected from the group consisting of absent, Glu and Arg;
Xaa₂is selected from the group consisting of absent, Gly, Phe, Lys, Asp, Glu, Ile, Arg and Cit;
Xaa₃ is selected from the group consisting of Asn, Asp, and Glu, wherein the alpha-amino group of Xaa₃ is optionally acylated with C₁-C₂₄ acyl;
Xaa₄ is Asn, pyrazolyl-alanine, or thiazolyl-alanine;
Xaa₅ is Ile;
Xaa₆ is the modified proline of formula (IV)

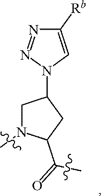

wherein R^{b} is phenyl substituted with R¹ or heteroaryl substituted with R^{l}, wherein R¹ is thienyl substituted with 1-2 C₁-C₆ alkyl groups;
Xaa₇ is Trp;
Xaa₈ is selected from the group consisting of Ser, Thr, 2,4-diaminobutanoic acid, Orn and Lys;
Xaa₉ is selected from the group consisting of absent, 2,4-diaminobutanoic acid, Orn, Lys, Glu, Glu-Ala, Glu-Ala-Met, Glu-Ala-Met-Met, and 2-(2-(2-aminoethoxy)ethoxy)acetic acid;
P₁ is absent, or is a group that comprises at least one thiol group and is covalently linked through an amide bond to (i) the C-terminus of Xaa₉ if Xaa₉ is not absent or (ii) the C-terminus of Xaa₈ if Xaa₉ is absent;
the side chain amino group of one residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉ forms an amide bond with the side chain carboxylic acid group of one residue selected from the group consisting of Glu at Xaa₂, Asp at Xaa₂, Glu at Xaa₃, and Asp at Xaa₃; and
the C-terminus of Xaa₈ is optionally amidated if Xaa₉ and P₁ are absent, or the C-terminus of Xaa₉ is optionally amidated if P₁ is absent.

Embodiment 32 provides the cyclic compound of any of Embodiments 29-31, wherein R^{b} is phenyl substituted with 2-thienyl which is substituted with at least one C₁-C₆ alkyl group.

Embodiment 33 provides the cyclic compound of Embodiment 32, wherein the C₁-C₆ alkyl group is methyl.

Embodiment 34 provides the cyclic compound of any of Embodiments 29-33, wherein Xaa₆ is

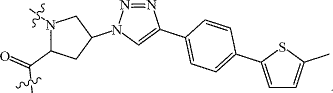

Embodiment 35 provides the cyclic compound of any of Embodiments 29-34, which is selected from the group consisting of:

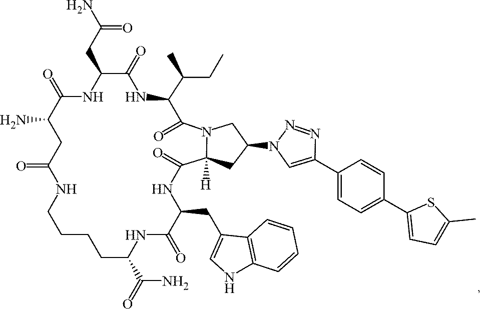

(3S,6S,14S,17S,20S,24S,25aS)-3-((1H-indol-3-yl)methyl)-14-amino-17-(2-amino-2-oxoethyl)-20-((S)-sec-butyl)-24-(4-(4-(5-methylthiophen-2-yl)phenyl)-1H-1,2,3-triazol-1-yl)-1,4,12,15,18,21-hexaoxotetracosahydro-1H-pyrrolo[2,1-f][1,4,7,10,13,18]hexaazacyclotricosine-6-carboxamide;

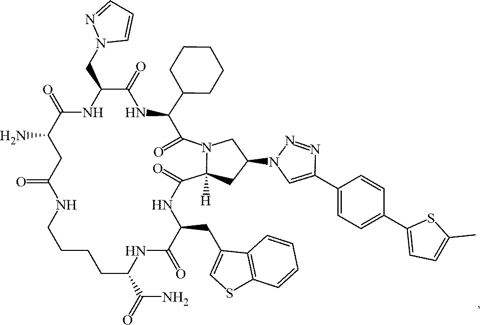

(3S,6S, 14S, 17S,20S,24S,25aS)-17-((1H-pyrazol-1-yl)methyl)-14-amino-3-(benzo[b]thiophen-3-ylmethyl)-20-cyclohexyl-24-(4-(4-(5-methylthiophen-2-yl)phenyl)-1H-1,2,3-triazol-1-yl)-1,4,12,15,18,21-hexaoxotetracosahydro-1H-pyrrolo[2,1-f][1,4,7,10,13,18]hexaazacyclotricosine-6-carboxamide;

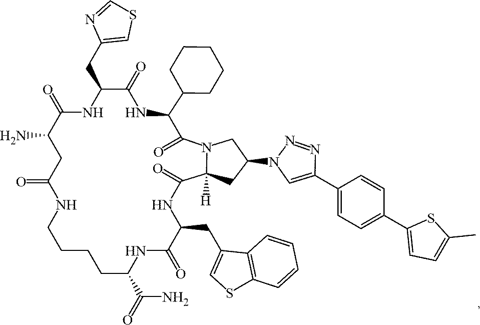

(3S,6S,14S,17S,20S,24S,25aS)-14-amino-3-(benzo[b]thiophen-3-ylmethyl)-20-cyclohexyl-24-(4-(4-(5-methylthiophen-2-yl)phenyl)-1H-1,2,3-triazol-1-yl)-1,4,12,15,18,21-hexaoxo-17-(thiazol-4-ylmethyl)tetracosahydro-1H-pyrrolo[2,1-f][1,4,7,10,13,18]hexaazacyclotricosine-6-carboxamide;

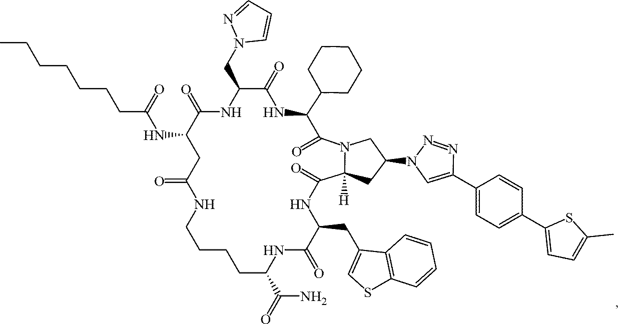

(3S,6S,14S,17S,20S,24S,25aS)-17-((1H-pyrazol-1-yl)methyl)-3-(benzo[b]thiophen-3-ylmethyl)-20-cyclohexyl-24-(4-(4-(5-methylthiophen-2-yl)phenyl)-1H-1,2,3-triazol-1-yl)-14-octanamido-1,4,12,15,18,21-hexaoxotetracosahydro-1H-pyrrolo[2,1-f][1,4,7,10,13,18]hexaazacyclotricosine-6-carboxamide; and

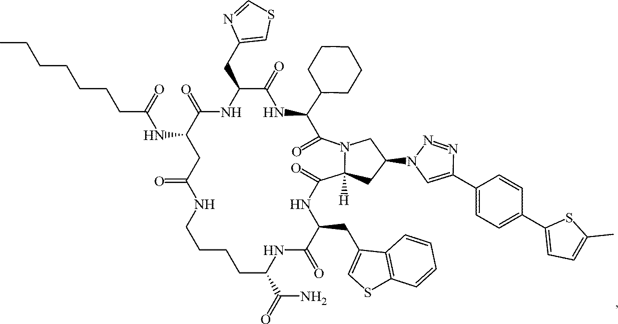

(35,68,1485,178,208,248S,25aS)-3-(benzo[b]thiophen-3-ylmethyl)-20-cyclohexyl-24-(4-(4-(5-methylthiophen-2-yl)phenyl)-1H-1,2,3-triazol-1-yl)-14-octanamido-1,4,12,15,18,21-hexaoxo-17-(thiazol-4-ylmethyl)tetracosahydro-1H-pyrrolo[2,1-f][1,4,7,10,13,18]hexaazacyclotricosine-6-carboxamide, or a salt or solvate thereof.

Embodiment 36 provides a pharmaceutical composition comprising at least one pharmaceutically acceptable carrier and at least one cyclic compound of any of Embodiments 29-35.

Embodiment 37 provides the composition of Embodiment 36, further comprising at least one additional compound useful for treating viral infections.

Embodiment 38 provides the composition of Embodiment 37, wherein the at least one additional compound is selected from the group consisting of antiviral combination drugs, entry and fusion inhibitors, integrase inhibitors, non-nucleoside reverse transcriptase inhibitors, nucleoside reverse transcriptase inhibitors, protease inhibitors, and any combinations thereof.

Embodiment 39 provides the composition of any of Embodiments 29-38, wherein the compound is encapsulated in a hydrogel and/or liposome.

Embodiment 40 provides the composition of Embodiment 39, wherein the hydrogel and/or liposome is pH-responsive.

Embodiment 41 provides the composition of any of Embodiments 39-40, wherein the hydrogel comprises a polymerized mixture of methacrylic acid and PEG-monomethyl ether monomethacrylate.

Embodiment 42 provides a method of treating, preventing, and/or ameliorating HIV-1 infection in a subject in need thereof, the method comprising administering to the subject a therapeutically effective amount of at least one cyclic compound of any one of Embodiments 29-35 or the pharmaceutical composition of any of Embodiments 36-41.

Embodiment 43 provides the method of Embodiment 42, wherein the subject is further administered at least one additional compound useful for treating viral infections.

Embodiment 44 provides the method of Embodiment 42 or 43, wherein the subject is a mammal.

Embodiment 45 provides the method of Embodiment 44, wherein the mammal is a human.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for promoting virolysis and/or inhibition of infection of a virus in a mammal, the composition comprising a lectin mutant selected from the group consisting of mutant cyanovirin N (CVN) and mutant Griffithsin (GRFT).

2. The composition of claim 1, wherein at least one of the following applies:
(a) the lectin mutant has at least 85% identity with CVN (SEQ ID NO:1) or GRFT (SEQ ID NO:2);
(b) the lectin mutant has at least 90% identity with CVN (SEQ ID NO:1) or GRFT (SEQ ID NO:2);
(c) the lectin mutant has at least 95% identity with CVN (SEQ ID NO:1) or GRFT (SEQ ID NO:2);
(d) the lectin mutant has at least 97.5% identity with CVN (SEQ ID NO:1) or GRFT (SEQ ID NO:2);
(e) the CVN mutant comprises a mutation at Pro51, optionally wherein the CVN mutant is P51G-CVN (SEQ ID NO:3).

3. (canceled)

4. (canceled)

5. (canceled)

6. (canceled)

7. The composition of claim 1, further comprising a S2 binding domain, wherein the lectin mutant and the binding domain are covalently linked by a flexible linker.

8. The composition of claim 7, wherein
the flexible linker comprises polyethylene glycol (PEG), or
the flexible linker comprises one or more amino acid residues.

9. (canceled)

10. The composition of claim 9, wherein the linker comprises one to ten instances of GGGGS (SEQ ID NO:4).

11. The composition of claim 10, wherein the linker comprises four instances of GGGGS (SEQ ID NO:4).

12. The composition of claim 7, wherein the binding domain comprises a HIV-1 MPER or MPER-like Trp3 domain.

13. The composition of claim 12, wherein the HIV-1 MPER or MPER-like Trp3 domain has at least 85% identity with HIV-1 MPER (SEQ ID NO:5) or MPER-like Trp3 domain (SEQ ID NO:6).

14. A pharmaceutical composition comprising at least one composition of claim 1 and at least one pharmaceutically acceptable carrier.

15. A method of treating, preventing, and/or ameliorating a viral infection in a subject, the method comprising administering to the subject a therapeutically effective amount of a composition comprising at least one composition of claim 1.

16. The method of claim 15, wherein at least one of the following applies:
(a) the viral infection is caused by a coronavirus, optionally the coronavirus is selected from the group consisting of SARS-CoV-1 and SARS-CoV-2;
(b) the viral infection is caused by HIV-1;
(c) the lectin is CVN (SEQ ID NO:1);
(d) the lectin is GRFT (SEQ ID NO:2);
(e) the lectin is P51G-CVN (SEQ ID NO:3);
(f) the composition comprises CVN-L4-Trp3;
(g) the subject is a mammal; or
(h) the subject is a human.

17. (canceled)

18. (canceled)

19. A method of promoting virolysis of a virus in a subject, the method comprising administering to the subject in need thereof a therapeutically effective amount of a composition comprising the composition of claim 1.

20. The method of claim 19, wherein at least one of the following applies:
(a) the virus is a coronavirus, optionally the coronavirus is selected from the group consisting of SARS-CoV-1 and SARS-CoV-2;
(b) the virus is HIV-1;
(c) the lectin is CVN (SEQ ID NO:1);
(d) the lectin is GRFT (SEQ ID NO:2);
(e) the lectin is P51G-CVN (SEQ ID NO:3);
(f) the composition comprises CVN-L4-Trp3;
(g) the subject is a mammal; or
(h) the subject is a human.

21. (canceled)

22. (canceled)

23. (canceled)

24. (canceled)

25. (canceled)

26. (canceled)

27. (canceled)

28. (canceled)

29. A cyclic compound of formula (I), or a salt, solvate, enantiomer or diastereoisomer thereof: 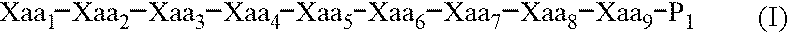
wherein in (I):
Xaa₁ is selected from the group consisting of absent, Glu and Arg;
Xaa₂ is selected from the group consisting of absent, Gly, Phe, Lys, Asp, Glu, Ile, Arg and Cit;
Xaa₃ is selected from the group consisting of absent, Asn, Asp, Ile, Glu and 2-cyclohexylglycine, wherein the alpha-amino group is optionally acylated with C₁-C₂₄ acyl;
Xaa₄ is selected from the group consisting of Asn, Asp, pyrazolyl-alanine, and thiazolyl-alanine;
Xaa₅ is a modified glycine of formula (III) 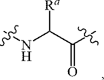
wherein R
^{a} is selected from the group consisting of C₁-C₆ alkyl and C₃-C₆ cycloalkyl;
Xaa₆ is the modified proline of formula (IV) 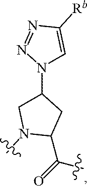
wherein R
^{b} is phenyl substituted with R¹ or heteroaryl substituted with R¹, wherein R¹ is thienyl substituted with 1-2 C₁-C₆ alkyl groups;
Xaa₇ is selected from the group consisting of Trp and 3-(3-benzothienyl)-L-alanine;
Xaa₈ is selected from the group consisting of Ser, Thr, 2,4-diaminobutanoic acid, Orn and Lys;
Xaa₉ is selected from the group consisting of absent, 2,4-diaminobutanoic acid, Orn, Lys, Glu, Glu-Ala, Glu-Ala-Met, Glu-Ala-Met-Met, and 2-(2-(2-aminoethoxy)ethoxy)acetic acid;
P₁ is absent, or is a group that comprises at least one thiol group and is covalently linked through an amide bond to (i) the C-terminus of Xaa₉ if Xaa₉ is not absent, or (ii) the C-terminus of Xaa₈ if Xaa₉ is absent;
the side chain amino group of one residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉ forms an amide bond with the side chain carboxylic acid group of one residue selected from the group consisting of Glu at Xaa₂, Asp at Xaa₂, Glu at Xaa₃, Asp at Xaa₃ and Asp at Xaa₄; and
the C-terminus of Xaa₈ is optionally amidated if Xaa₉ and P₁ are absent, or the C-terminus of Xaa₉ is optionally amidated if P₁ is absent.

30. The cyclic compound of claim 29, wherein at least one of the following applies:
(a) the cyclic compound is selected from the group consisting of: 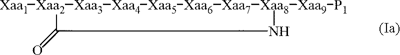 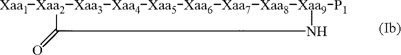 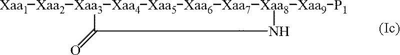 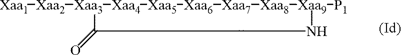 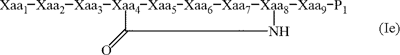 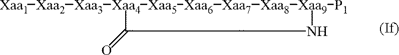
wherein in (Ia)-(If):
‘NH’ is derived from the side chain amino group of a residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉, and
‘C═O’ is derived from the side chain carboxylic acid group of a residue selected from the group consisting of Glu at Xaa₂, Asp at Xaa₂, Glu at Xaa₃, Asp at Xaa₃, and Asp at Xaa₄, or
(b) the cyclic compound is a cyclic compound of formula (II): 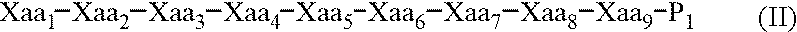
wherein in (II):
Xaa₁ is selected from the group consisting of absent, Glu and Arg;
Xaa₂ is selected from the group consisting of absent, Gly, Phe, Lys, Asp, Glu, Ile, Arg and Cit;
Xaa₃ is selected from the group consisting of Asn, Asp, and Glu, wherein the alpha-amino group of Xaa₃ is optionally acylated with C₁-C₂₄ acyl;
Xaa₄ is Asn, pyrazolyl-alanine, or thiazolyl-alanine;
Xaa₅ is Ile_{:}
Xaa₆ is the modified proline of formula (IV) 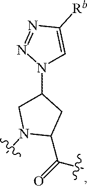
wherein R
^{b} is phenyl substituted with R¹ or heteroaryl substituted with R¹, wherein R¹ is thienyl substituted with 1-2 C₁-C₆ alkyl groups;
Xaa₇ is Trp;
Xaa₈ is selected from the group consisting of Ser, Thr, 2,4-diaminobutanoic acid, Orn and Lys;
Xaa₉ is selected from the group consisting of absent, 2,4-diaminobutanoic acid, Orn, Lys, Glu, Glu-Ala, Glu-Ala-Met, Glu-Ala-Met-Met, and 2-(2-(2-aminoethoxy)ethoxy)acetic acid;
P₁ is absent, or is a group that comprises at least one thiol group and is covalently linked through an amide bond to (i) the C-terminus of Xaa₉ if Xaa₉ is not absent or (ii) the C-terminus of Xaa₈ if Xaa₉ is absent;
the side chain amino group of one residue selected from the group consisting of 2,4-diaminobutanoic acid at Xaa₈, Orn at Xaa₈, Lys at Xaa₈, 2,4-diaminobutanoic acid at Xaa₉, Orn at Xaa₉, and Lys at Xaa₉ forms an amide bond with the side chain carboxylic acid group of one residue selected from the group consisting of Glu at Xaa₂, Asp at Xaa₂, Glu at Xaa₃, and Asp at Xaa₃, and
the C-terminus of Xaa₈ is optionally amidated if Xaa₉ and P₁ are absent, or the C-terminus of Xaa₉ is optionally amidated if P₁ is absent.

31. (canceled)

32. The cyclic compound of claim 29, wherein at least one of the following applies:
(a) R^{b} is phenyl substituted with 2-thienyl which is substituted with at least one C₁-C₆ alkyl group, optionally C₁-C₆ alkyl group is methyl;
(b) Xaa₆ is 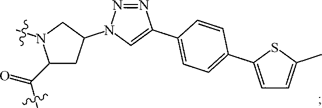
(c) the cyclic compound is selected from the group consisting of: 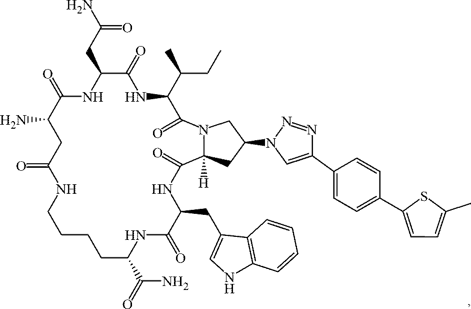
(3S,6S,14S,17S,20S,24S,25aS)-3-((1H-indol-3-yl)methyl)-14-amino-17-(2-amino-2-oxoethyl)-20-((S)-sec-butyl)-24-(4-(4-(5-methylthiophen-2-yl)phenyl)-1H-1,2,3-triazol-1-yl)-1,4,12,15,18,21-hexaoxotetracosahydro-1H-pyrrolo[2,1-f][1,4,7,10,13,18]hexaazacyclotricosine-6-carboxamide, 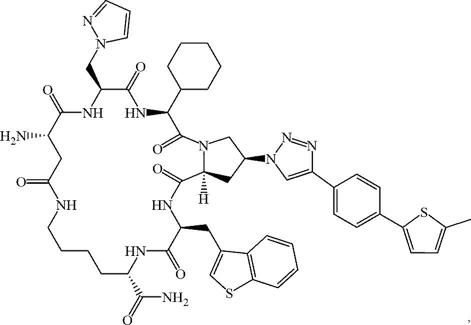
(3S,6S,14S,17S,20S,24S,25aS)-17-((1H-pyrazol-1-yl)methyl)-14-amino-3-(benzo[b]lthiophen-3-ylmethyl)-20-cyclohexyl-24-(4-(4-(5-methylthiophen-2-yl)phenyl)-1H-1,2,3-triazol-1-yl)-1,4,12,15,18,21-hexaoxotetracosahydro-1H-pyrrolo[2,1-f][1,4,7,10,13,18]hexaazacyclotricosine-6-carboxamide; 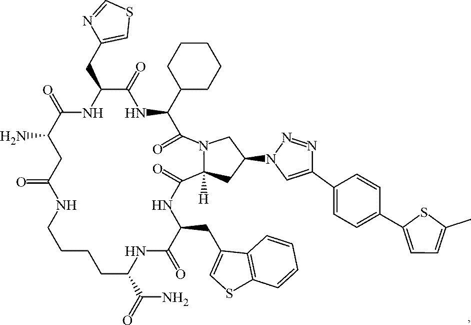
(3S,6S,14S,17S,20S,24S,25aS)-14-amino-3-(benzo[b]thiophen-3-ylmethyl)-20-cyclohexyl-24-(4-(4-(5-methylthiophen-2-yl)phenyl)-1H-1,2,3-triazol-1-yl)-1,4,12,15,18,21-hexaoxo-17-(thiazol-4-ylmethyl)tetracosahydro-1H-pyrrolo[2,1-f][1,4,7,10,13,18]hexaazacyclotricosine-6-carboxamide; 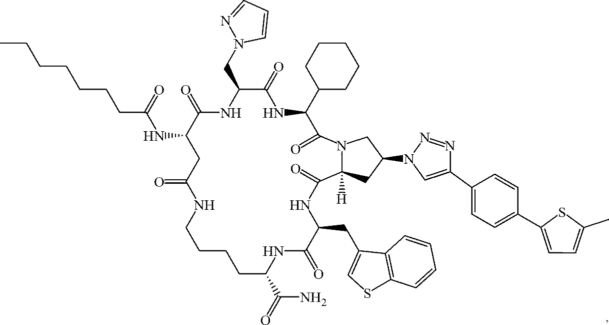
(3S,6S,14S,17S,20S,24S,25aS)-17-((1H-pyrazol-1-yl)methyl)-3-(benzo[b]thiophen-3-ylmethyl)-20-cyclohexyl-24-(4-(4-(5-methylthiophen-2-yl)phenyl)-1H-1,2,3-triazol-1-yl)-14-octanamido-1,4,12,15,18,21-hexaoxotetracosahydro-1H-pyrrolo[2,1-f][1,4,7,10,13,18]hexaazacyclotricosine-6-carboxamide; and 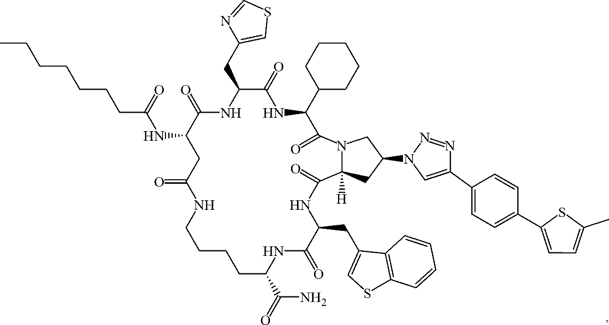
(3S,6S,14S,17S,20S,24S,25aS)-3-(benzo[b]thiophen-3-ylmethyl)-20-cyclohexyl-24-(4-(4-(5-methylthiophen-2-yl)phenyl)-1H-1,2,3-triazol-1-yl)-14-octanamido-1,4,12,15,18,21-hexaoxo-17-(thiazol-4-ylmethyl)tetracosahydro-1H-pyrrolo[2,1-f][1,4,7,10,13,18]hexaazacyclotricosine-6-carboxamide, or a salt or solvate thereof.

33. (canceled)

34. (canceled)

35. (canceled)

36. A pharmaceutical composition comprising at least one pharmaceutically acceptable carrier and the cyclic compound of claim 29.

37. The composition of claim 36, wherein at least one of the following applies:
(a) the composition further comprises at least one additional compound useful for treating viral infections, optionally the at least one additional compound is selected from the group consisting of antiviral combination drugs, entry and fusion inhibitors, integrase inhibitors, non-nucleoside reverse transcriptase inhibitors, nucleoside reverse transcriptase inhibitors, protease inhibitors, and any combinations thereof; or
(b) the compound is encapsulated in a hydrogel and/or liposome, optionally the hydrogel and/or liposome is pH-responsive, optionally the hydrogel comprises a polymerized mixture of methacrylic acid and PEG-monomethyl ether monomethacrylate.

38. (canceled)

39. (canceled)

40. (canceled)

41. (canceled)

42. A method of treating, preventing, and/or ameliorating HIV-1 infection in a subject in need thereof, the method comprising administering to the subject a therapeutically effective amount of at least one cyclic compound of claim 29.

43. The method of claim 42, wherein at least one of the following applies:
(a) the subject is further administered at least one additional compound useful for treating viral infections;
(b) the subject is a mammal;
(c) the subject is a human.

44. (canceled)

45. (canceled)


SEQUENCE LISTING (6 sequences)

SEQ ID NO: 1 - protein, 101 aa, Artificial Sequence
  Chemically synthesized
LGKFSQTCYNSAIQGSVLTSTCERTNGGYNTSSIDLNSVIENVDGSLKWQPSNFIETCRNTQLAGSSELAAECKTRAQQFVSTKINLDDHIANIDGTLKYE

SEQ ID NO: 2 - protein, 121 aa, Artificial Sequence
  Chemically synthesized
  misc_feature (31)..(31): Xaa can be any naturally occurring amino acid
SLTHRKFGGSGGSPFSGLSSIAVRSGSYLDXIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 3 - protein, 101 aa, Artificial Sequence
  Chemically synthesized
LGKFSQTCYNSAIQGSVLTSTCERTNGGYNTSSIDLNSVIENVDGSLKWQGSNFIETCRNTQLAGSSELAAECKTRAQQFVSTKINLDDHIANIDGTLKYE

SEQ ID NO: 4 - protein, 5 aa, Artificial Sequence
  Chemically synthesized
GGGGS

SEQ ID NO: 5 - protein, 20 aa, Artificial Sequence
  Chemically synthesized
DKWASLWNWFNITNWLWYIK

SEQ ID NO: 6 - protein, 9 aa, Artificial Sequence
  Chemically synthesized
DKWASLWNW